(19) United States
(12) Patent Application Publication
(10) Pub. No.: US 2023/0204581 A1
(43) Pub. Date: Jun. 29, 2023

(54) COMPOSITIONS AND METHODS FOR THE DETECTION OF VIRUSES IN A BIOLOGICAL SAMPLE

(71) Applicant: Biomerica, Inc., Irvine, CA (US)

(72) Inventors: Elisabeth Laderman, Irvine, CA (US); Zackary Irani-Cohen, Irvine, CA (US)

(21) Appl. No.: 17/822,431

(22) Filed: Aug. 26, 2022

Related U.S. Application Data
(63) Continuation of application No. PCT/IB2021/051784, filed on Mar. 3, 2021.
(60) Provisional application No. 63/011,299, filed on Apr. 17, 2020.
(60) Provisional application No. 63/011,289, filed on Apr. 16, 2020.
(60) Provisional application No. 62/990,966, filed on Mar. 17, 2020.
(60) Provisional application No. 62/991,048, filed on Mar. 17, 2020.
(60) Provisional application No. 62/984,774, filed on Mar. 3, 2020.

(51) Int. Cl. G01N 33/569; G01N 33/543

(52) U.S. Cl. CPC G01N 33/56983; G01N 33/54388; G01N 2469/10; G01N 2333/165

(57) ABSTRACT

The disclosure provides compositions and methods for detecting coronaviruses, in particular SARS-CoV-2, in biological samples. The disclosure also provides compositions and methods for simultaneously detecting coronaviruses and influenzaviruses in biological samples.

### CROSS-REFERENCE TO RELATED APPLICATIONS

This application is a continuation of International Application No. PCT/IB2021/051784, filed on Mar. 3, 2021, which in turn claims the benefit of priority to U.S. Provisional Application No. 62/984,774, filed on Mar. 3, 2020; U.S. Provisional Application No. 62/990,966, filed on Mar. 17, 2020; U.S. Provisional Application No. 62/991,048, filed on Mar. 17, 2020; U.S. Provisional Application No. 63/011,289, filed on Apr. 16, 2020; and U.S. Provisional Application No. 63/011,299, filed on Apr. 17, 2020. The entire contents of the foregoing applications are hereby incorporated herein by reference.

### BACKGROUND OF THE INVENTION

This disclosure relates to compositions and methods for detecting coronaviruses, (e.g., SARS-CoV-2, 229E, NL63, OC43, HKU1, MERS-CoV and SARS-CoV) in a sample, e.g., a biological sample (e.g., an oral sample, a nasal sample, a fecal sample, and/or a sputum sample). This disclosure also relates to compositions and methods for detecting coronaviruses and influenza viruses (e.g., influenzavirus A, influenzavirus B, influenzavirus C, and influenzavirus D) in a sample, e.g., a biological sample.

Coronaviruses are named for the crown-like spikes on their surface. There are four main sub-groupings of coronaviruses, known as alpha, beta, gamma, and delta. Human coronaviruses were first identified in the mid-1960s. There are several coronaviruses that can infect people, including, but not limited to, 229E (alpha coronavirus), NL63 (alpha coronavirus), OC43 (beta coronavirus), HKU1 (beta coronavirus), MERS-CoV (the beta coronavirus that causes Middle East Respiratory Syndrome, or MERS), SARS-CoV (the beta coronavirus that causes severe acute respiratory syndrome, or SARS) and SARS-CoV-2 (the novel coronavirus that causes coronavirus disease 2019, or COVID-19). People around the world commonly get infected with human coronaviruses 229E, NL63, OC43, and HKU1. However, sometimes coronaviruses that infect animals can evolve and make people sick and become a new human coronavirus. Recent examples of new human coronaviruses are SARS-CoV-2, 2019-nCoV, SARS-CoV, and MERS-CoV.

SARS-CoV-2 is a virus that emerged in China in late 2019 and was isolated from the respiratory epithelium of patients as first described in 2020 by Xu et a (Viruses 2020, 12, 244; doi:10.3390/v12020244). SARS-CoV-2 belongs to a family of viruses, the Coronaviridae, a group IV ((+) ssRNA) virus of the genus betacoronavirus following the nomenclature of the Coronavirus Study group (de Groot 2013). Coronaviruses are generally zoonotic, meaning they are transmitted between animals and people. The complete genome of the human SARS-CoV-2 virus has been deposited under the GenBank accession number MN908947.3. The SARS-CoV-2 outbreak has been declared a public health emergency of international concern by the World Health Organization, causing significant impact on people's lives, families and communities.

Influenza viruses (types A, B, and C) are members of the orthomyxoviridae family that cause influenza. Type A influenza viruses infect birds and mammals, including humans, whereas types B and C only infect humans. Influenza viruses are roughly spherical enveloped viruses of about 8-200 nm diameter that contain segmented negative sense genomic RNA. Human influenza viruses produce highly contagious, acute respiratory disease that results in significant morbidity and economic costs, with significant mortality among very young, elderly, and immuno-compromised subpopulations. It is estimated that influenza accounts for approximately 500,000 hospitalizations and 38,000 deaths in the United States each year.

Accordingly, a need exists for compositions and methods for accurately and efficiently detecting SARS-CoV-2. A need also exists for the development of rapid diagnostic tests that can detect and distinguish between coronavirus and influenza in a patient clinical sample accurately with efficiency at point-of-care in a relatively short time, with a minimum of exposure of technical personnel to infectious agents, so that a diagnosis is completed in sufficient time to permit effective therapeutic treatment of an infected person.

### SUMMARY OF THE INVENTION

The present disclosure provides compositions and methods for the rapid detection of coronavirus, and in particular, SARS-CoV-2 antigens in a biological sample. The present disclosure also provides compositions and methods for the simultaneous or discrete rapid detection of coronavirus (e.g., SARS-CoV-2) and influenzavirus, in a single test kit.

In one aspect, the present disclosure provides a rapid detection test system for the detection of SARS-CoV-2 or a variant thereof. The rapid detection system comprises a sample pad comprising a porous material, a conjugate pad comprising a solid support, and a membrane comprising a test zone and a control zone. In various embodiments of the system, the conjugate pad comprises a first anti-SARS-CoV-2 antibody attached to the solid support to form a mobilizable conjugate. The first anti-SARS-CoV-2 antibody of the mobilizable conjugate is capable of binding to a SARS-CoV-2 antigen to form a mobilizable conjugate-antigen complex. In various embodiments of the system, the test zone comprises a second anti-SARS-CoV-2 antibody immobilized to the membrane. The second anti-SARS-CoV-2 antibody is capable of binding to the mobilizable conjugate-antigen complex to form a first immobilizable complex. In various embodiments of the system, the control zone comprises an immunoglobulin immobilized to the membrane. The immunoglobulin is capable of binding to the mobilizable conjugate to form a second immobilizable complex.

In one embodiment, the SARS-CoV-2 antigen is selected from the group consisting of a spike protein (S), a receptor-binding (RBD) protein, a S1 protein, a S2 protein, a whole protein (S1+S2), and a nucleocapsid protein (NP). In a specific embodiment, the SARS-CoV-2 antigen is a nucleocapsid protein. In another specific embodiment, the SARS-CoV-2 antigen is a spike protein.

In one embodiment, the sample is selected from the group consisting of oral fluid, saliva, blood, blood plasma, blood serum, nasopharyngeal fluid, amniotic fluid, breast milk, vaginal secretions, semen, seminal fluid, urine, amniotic fluid, cerebrospinal fluid, bronchoalveolar lavage fluid, tears, mucous, tissue, tissue homogenate, cellular extract and spinal fluid. In a specific embodiment, the sample is saliva. In another specific embodiment, the sample is blood. In various embodiments described above, the sample is dispersed in a sample buffer. In one embodiment, the sample buffer comprises a phosphate buffered saline solution.

In various embodiments described above, the system further comprises a conjugation reaction buffer. In one embodiment, the conjugation reaction buffer comprises about 5 mM Potassium Phosphate, about 5 mg/mL PEG20, and wherein the buffer has a pH between about 7.0-7.5. In various embodiments described above, the system further comprises a blocking buffer. In one embodiment, the blocking buffer comprises about 4 mM borate and about 1% BSA.

In one embodiment, the sample pad comprises a porous material. The porous material comprises a matrix selected from the group consisting of a glass fiber, a nitrocellulose, a nitrocellulose blend with polyester, a nitrocellulose blend with cellulose, an untreated paper, a porous paper, and an acrylonitrile copolymer. In one embodiment, the porous material comprises a matrix comprising a glass fiber. In one embodiment, the sample pad is pretreated with a buffered solution. In some embodiments, the sample pad is pretreated with a buffered solution comprising phosphate buffered saline (PBS). In one embodiment, the conjugate pad comprises a matrix comprising a glass fiber. In various embodiments described above, the solid support comprises a gold nanoparticle. In one embodiment, the membrane is a nitrocellulose membrane.

In many embodiments, the conjugate pad is prepared by loading at least about 15 μg/mL of the first anti-SARS-CoV-2 antibody (i.e., 15 μg of the first anti-SARS-CoV-2 antibody per 1 mL of the solid support) onto the conjugate pad. In other embodiments, the conjugate pad is prepared by loading at least about 25 μg/mL of the first anti-SARS-CoV-2 antibody (i.e., 25 μg of the first anti-SARS-CoV-2 antibody per 1 mL of the solid support) onto the conjugate pad. In some embodiments, the conjugate pad comprises not more than about 10 μL of OD₂₀ mobilizable conjugate.

In various embodiments described above, the first anti-SARS-CoV-2 antibody is Antibody B (Clone B3451M). In some embodiments, the second anti-SARS-CoV-2 antibody is Antibody A (Clone B3449M). In one embodiment, the first anti-SARS-CoV-2 antibody is Antibody B (Clone B3451M) and the second anti-SARS-CoV-2 antibody is Antibody A (Clone B3449M).

In another aspect, the present disclosure provides a kit for the detection of SARS-CoV-2 comprising a rapid detection test system as described herein, and reagents and/or instructions for use.

In another aspect, the present disclosure provides a method for the rapid detection of SARS-CoV-2, or a variant thereof, in a biological sample. The method comprises the steps of a) dispersing a biological sample suspected of having a SARS-CoV-2 antigen in a sample buffer; b) contacting the biological sample dispersed in the sample buffer with a sample pad comprising a porous material through which the biological sample can flow by capillary action, c) contacting the sample of step b) with a conjugate pad such that the contacting is performed under conditions that permit the capillarity of a mobilizable conjugate formed into conjugate pad. The conjugate pad comprises a solid support to which a first anti-SARS-CoV-2 antibody is attached, forming a mobilizable conjugate. The first anti-SARS-CoV-2 antibody of the mobilizable conjugate is capable of binding to the SARS-CoV-2 antigen to form a mobilizable conjugate-antigen complex. The method further comprises the steps of contacting the mobilizable conjugate-antigen complex with a membrane comprising a test zone. The test zone comprises a second anti-SARS-CoV-2 antibody immobilized to the membrane. The second anti-SARS-CoV-2 antibody is capable of binding to the mobilizable conjugate-antigen complex to form a first immobilizable complex. The first immobilizable complex produces a detectable signal at the test zone indicating the presence of the SARS-CoV-2 antigen in the biological sample.

In one embodiment of the method, the second anti-SARS-CoV-2 antibody is conjugated to a detectable agent capable of producing a detectable signal.

In one embodiment of the method, the membrane further comprises a control zone downstream of the test zone. The control zone comprises an immunoglobulin immobilized to the membrane. The immunoglobulin is capable of binding to the mobilizable conjugate to form a second immobilizable complex. The second immobilizable complex produces a detectable signal at the control zone indicating capillarity of a biological sample.

In various embodiments of the method described above, the detectable signal is capable of visual detection without instrumentation.

In various embodiments of the method, the SARS-CoV-2 antigen is selected from the group consisting of a spike protein (S), a receptor-binding (RBD) protein, a S1 protein, a S2 protein, a whole protein (S1+S2), and a nucleocapsid protein (NP). In one embodiment, the SARS-CoV-2 antigen is a nucleocapsid protein. In another embodiment, the SARS-CoV-2 antigen is a spike protein.

In many embodiments of the method, the sample is selected from the group consisting of oral fluid, saliva, blood, blood plasma, blood serum, nasopharyngeal fluid, amniotic fluid, breast milk, vaginal secretions, semen, seminal fluid, urine, amniotic fluid, cerebrospinal fluid, bronchoalveolar lavage fluid, tears, mucous, tissue, tissue homogenate, cellular extract and spinal fluid. In one embodiment, the biological sample is an oral specimen. In a specific embodiment, the biological sample is saliva. In another embodiment, the biological sample is a nasal specimen. In another specific embodiment, the sample is blood. In various embodiments described above, the sample is dispersed in a sample buffer. In one embodiment, the sample buffer comprises a phosphate buffered saline solution.

In various embodiments, method described above further comprises a pretreatment step. The pretreatment step comprises contacting the sample pad with a phosphate buffered saline solution. In various embodiments, method described above further comprises a blocking step. The blocking step comprises contacting the conjugation pad with a blocking buffer for at least 30 minutes prior to step b). In one embodiment, the blocking buffer comprises about 4 mM borate and about 1% BSA.

In various embodiments of the method described above, the first anti-SARS-CoV-2 antibody is Antibody B (Clone B3451M). In some embodiments, the second anti-SARS-CoV-2 antibody is Antibody A (Clone B3449M). In one embodiment, the first anti-SARS-CoV-2 antibody is Antibody B (Clone B3451M) and the second anti-SARS-CoV-2 antibody is Antibody A (Clone B3449M).

In certain embodiments of the method described above, the conjugate pad is prepared by loading at least about 15 μg/mL of the first anti-SARS-CoV-2 antibody onto the conjugate pad. In other embodiments, the conjugate pad is prepared by loading at least about 25 μg/mL of the first anti-SARS-CoV-2 antibody onto the conjugate pad. In some embodiments, the conjugate pad comprises not more than about 10 μL of OD₂₀ mobilizable conjugate.

In various embodiments of the method described above, the second anti-SARS-CoV-2 antibody is immobilized on the membrane at a concentration of between about 10 μg/mL to 30 μg/mL. In one embodiment, the second anti-SARS-CoV-2 antibody is immobilized on the membrane at a concentration of about 15 μg/mL. In another embodiment, the first anti-SARS-CoV-2 antibody is immobilized on the solid support at a concentration of about 15 μg/mL and the second anti-SARS-CoV-2 antibody is immobilized on the membrane at a concentration of about 15 μg/mL.

In various embodiments of the method described above, the membrane is a nitrocellulose membrane.

In another aspect, the present disclosure provides a rapid detection test system for the detection of coronavirus, or a variant thereof. The rapid detection system comprises a sample pad comprising a porous material, a conjugate pad comprising a solid support, and a membrane comprising a test zone and a control zone. In various embodiments of the system, the conjugate pad comprises a first anti-coronavirus antibody is attached to the solid support to form a mobilizable conjugate. The first anti-coronavirus antibody of the mobilizable conjugate is capable of binding to a coronavirus antigen to form a mobilizable conjugate-antigen complex. In various embodiments of the system, the test zone comprises a second anti-coronavirus antibody immobilized to the membrane. The second anti-coronavirus antibody is capable of binding to the mobilizable conjugate-antigen complex to form a first immobilizable complex. In various embodiments of the system, the control zone comprises an immunoglobulin immobilized to the membrane. The immunoglobulin is capable of binding to the mobilizable conjugate to form a second immobilizable complex.

In one embodiment, the first anti-coronavirus antibody is capable of binding to at least one coronavirus selected from the group consisting of 229E, NL63, OC43, HKU1, MERS-CoV, SARS-CoV and SARS-CoV-2. In another embodiment, the second anti-coronavirus antibody is capable of binding to at least one coronavirus selected from the group consisting of 229E, NL63, OC43, HKU1, MERS-CoV, SARS-CoV and SARS-CoV-2. In some embodiments, the coronavirus antigen described in the method to detect a coronavirus is selected from the group consisting of a spike protein (S), a receptor-binding (RBD) protein, a S1 protein, a S2 protein, a whole protein (S1+S2), and a nucleocapsid protein (NP). In a specific embodiment, the coronavirus antigen is a nucleocapsid protein. In another specific embodiment, the coronavirus antigen is a spike protein.

In one embodiment, the sample is selected from the group consisting of oral fluid, saliva, blood, blood plasma, blood serum, nasopharyngeal fluid, amniotic fluid, breast milk, vaginal secretions, semen, seminal fluid, urine, amniotic fluid, cerebrospinal fluid, bronchoalveolar lavage fluid, tears, mucous, tissue, tissue homogenate, cellular extract and spinal fluid. In a specific embodiment, the sample is saliva. In another specific embodiment, the sample is blood. In various embodiments described above, the sample is dispersed in a sample buffer. In one embodiment, the sample buffer comprises a phosphate buffered saline solution.

In various embodiments described above, the system further comprises a conjugation reaction buffer. In one embodiment, the conjugation reaction buffer comprises about 5 mM Potassium Phosphate, about 5 mg/mL PEG20, and wherein the buffer has a pH between about 7.0-7.5. In various embodiments described above, the system further comprises a blocking buffer. In one embodiment, the blocking buffer comprises about 4 mM borate and about 1% BSA.

In one embodiment, the sample pad comprises a porous material. The porous material comprises a matrix selected from the group consisting of a glass fiber, a nitrocellulose, a nitrocellulose blend with polyester, a nitrocellulose blend with cellulose, an untreated paper, a porous paper, and an acrylonitrile copolymer. In one embodiment, the porous material comprises a matrix comprising a glass fiber. In one embodiment, the sample pad is pretreated with a buffered solution. In some embodiments, the sample pad is pretreated with a buffered solution comprising phosphate buffered saline (PBS). In one embodiment, the conjugate pad comprises a matrix comprising a glass fiber. In various embodiments described above, the solid support comprises a gold nanoparticle. In one embodiment, the membrane is a nitrocellulose membrane.

In many embodiments, the conjugate pad is prepared by loading at least about 15 μg/mL of the first anti-coronavirus antibody (i.e., 15 μg of the first anti-coronavirus antibody per 1 mL of the solid support) onto the conjugate pad. In other embodiments, the conjugate pad is prepared by loading at least about 25 μg/mL of the first anti-coronavirus antibody (i.e., 25 μg of the first anti-coronavirus antibody per 1 mL of the solid support) onto the conjugate pad. In some embodiments, the conjugate pad comprises not more than about 10 μL of OD₂₀ mobilizable conjugate.

In another aspect, the present disclosure provides a kit for the detection of a coronavirus comprising a rapid detection test system as described herein, and reagents and/or instructions for use.

In a related aspect, the present disclosure provides a method for the rapid detection of a coronavirus, or a variant thereof, in a biological sample. The method comprises the steps of a) dispersing a biological sample suspected of having a coronavirus antigen in a sample buffer; b) contacting the biological sample dispersed in the sample buffer with a sample pad comprising a porous material through which the biological sample can flow by capillary action, c) contacting the sample of step b) with a conjugate pad such that the contacting is performed under conditions that permit the capillarity of a mobilizable conjugate formed into conjugate pad. The conjugate pad comprises a solid support to which a first anti-coronavirus antibody is attached, forming a mobilizable conjugate. The first anti-coronavirus antibody of the mobilizable conjugate is capable of binding to the coronavirus antigen to form a mobilizable conjugate-antigen complex. The method further comprises the steps of contacting the mobilizable conjugate-antigen complex with a membrane comprising a test zone. The test zone comprises a second anti-coronavirus antibody immobilized to the membrane. The second anti-coronavirus antibody is capable of binding to the mobilizable conjugate-antigen complex to form a first immobilizable complex. The first immobilizable complex produces a detectable signal at the test zone indicating the presence of the coronavirus antigen in the biological sample.

In one embodiment of the method, the second anti-coronavirus antibody is conjugated to a detectable agent capable of producing a detectable signal.

In one embodiment of the method, the membrane further comprises a control zone downstream of the test zone. The control zone comprises an immunoglobulin immobilized to the membrane. The immunoglobulin is capable of binding to the mobilizable conjugate to form a second immobilizable complex. The second immobilizable complex produces a detectable signal at the control zone indicating capillarity of a biological sample.

In various embodiments of the method described above, the detectable signal is capable of visual detection without instrumentation.

In various embodiments of the method, the coronavirus antigen is selected from the group consisting of a spike protein (S), a receptor-binding (RBD) protein, a S1 protein, a S2 protein, a whole protein (S1+S2), and a nucleocapsid protein (NP). In one embodiment, the coronavirus antigen is a nucleocapsid protein. In another embodiment, the coronavirus antigen is a spike protein.

In many embodiments of the method, the sample is selected from the group consisting of oral fluid, saliva, blood, blood plasma, blood serum, nasopharyngeal fluid, amniotic fluid, breast milk, vaginal secretions, semen, seminal fluid, urine, amniotic fluid, cerebrospinal fluid, bronchoalveolar lavage fluid, tears, mucous, tissue, tissue homogenate, cellular extract and spinal fluid. In one embodiment, the biological sample is an oral specimen. In a specific embodiment, the biological sample is saliva. In another embodiment, the biological sample is a nasal specimen. In another specific embodiment, the sample is blood. In various embodiments described above, the sample is dispersed in a sample buffer. In one embodiment, the sample buffer comprises a phosphate buffered saline solution.

In various embodiments, method described above further comprises a pretreatment step. The pretreatment step comprises contacting the sample pad with a phosphate buffered saline solution. In various embodiments, method described above further comprises a blocking step. The blocking step comprises contacting the conjugation pad with a blocking buffer for at least 30 minutes prior to step b). In one embodiment, the blocking buffer comprises about 4 mM borate and about 1% BSA.

In certain embodiments of the method described above, the conjugate pad is prepared by loading at least about 15 μg/mL of the first anti-coronavirus antibody onto the conjugate pad. In other embodiments, the conjugate pad is prepared by loading at least about 25 μg/mL of the first anti-coronavirus antibody onto the conjugate pad. In some embodiments, the conjugate pad comprises not more than about 10 μL of OD₂₀ mobilizable conjugate.

In various embodiments of the method described above, the second anti-coronavirus antibody is immobilized on the membrane at a concentration of between about 10 μg/mL to 30 μg/mL. In one embodiment, the second anti-coronavirus antibody is immobilized on the membrane at a concentration of about 15 μg/mL. In another embodiment, the first anti-coronavirus antibody is immobilized on the solid support at a concentration of about 15 μg/mL and the second anti-coronavirus antibody is immobilized on the membrane at a concentration of about 15 μg/mL.

In various embodiments of the method described above, the membrane is a nitrocellulose membrane.

In yet another aspect, the present disclosure provides a rapid detection test system for the detection of a coronavirus and an influenzavirus. The rapid detection system comprises a sample pad comprising a porous material, a conjugate pad, and a membrane comprising a first test zone, a second test zone, and at least one control zone. In various embodiments of the system, the conjugate pad comprises a first anti-coronavirus antibody is attached to a first solid support to form a first mobilizable conjugate. The first anti-coronavirus antibody of the first mobilizable conjugate is capable of binding to a coronavirus antigen to form a first mobilizable conjugate-antigen complex. The conjugate pad further comprises a first anti-influenzavirus antibody is attached to a second solid support to form a second mobilizable conjugate. The first anti-influenzavirus antibody of the second mobilizable conjugate is capable of binding to influenzavirus antigen to form a second mobilizable conjugate-antigen complex.

In various embodiments of the system, the first test zone comprises a second anti-coronavirus antibody immobilized to the membrane. The second anti-coronavirus antibody is capable of binding to the first mobilizable conjugate-antigen complex to form a first immobilizable complex. The second test zone comprises a second anti-influenzavirus antibody immobilized to the membrane. The second anti-influenzavirus antibody is capable of binding to the second mobilizable conjugate-antigen complex to form a second immobilizable complex. The at least one control zone comprises an immunoglobulin immobilized to the membrane. The immunoglobulin is capable of binding to the first mobilizable conjugate to form a third immobilizable complex, and the immunoglobulin is further capable of binding to the second mobilizable conjugate to form a fourth immobilizable complex.

In one embodiment, the membrane comprises a first control zone and a second control zone. The first control zone is downstream of the first test zone, and the second control zone is downstream of the second test zone. The first control zone comprises a first immunoglobulin immobilized to the membrane such that the immunoglobulin is capable of binding to the first mobilizable conjugate to form the third immobilizable complex. The second control zone comprises a second immunoglobulin immobilized to the membrane such that the second immunoglobulin is capable of binding to the second mobilizable conjugate to form the fourth immobilizable complex. The third immobilizable complex and the fourth immobilizable complex produce a detectable signal at the first and second control zones, respectively, indicating capillarity of the biological sample.

In one embodiment, the first anti-coronavirus antibody is capable of binding to at least one coronavirus selected from the group consisting of 229E, NL63, OC43, HKU1, MERS-CoV, SARS-CoV and SARS-CoV-2. In another embodiment, the second anti-coronavirus antibody is capable of binding to at least one coronavirus selected from the group consisting of 229E, NL63, OC43, HKU1, MERS-CoV, SARS-CoV and SARS-CoV-2. In some embodiments, the coronavirus antigen described in the method to detect a coronavirus is selected from the group consisting of a spike protein (S), a receptor-binding (RBD) protein, a S1 protein, a S2 protein, a whole protein (S1+S2), and a nucleocapsid protein (NP). In a specific embodiment, the coronavirus antigen is a nucleocapsid protein. In another specific embodiment, the coronavirus antigen is a spike protein.

In one embodiment, the first anti-influenzavirus antibody is capable of binding to at least one influenzavirus antigen selected from the group viruses consisting of influenzavirus A, influenzavirus B, influenzavirus C, and influenzavirus D. In another embodiment, the second anti-influenzavirus antibody is capable of binding to at least one influenzavirus antigen selected from the group viruses consisting of influenzavirus A, influenzavirus B, influenzavirus C, and influenzavirus D.

In one embodiment, the sample is selected from the group consisting of oral fluid, saliva, blood, blood plasma, blood serum, nasopharyngeal fluid, amniotic fluid, breast milk, vaginal secretions, semen, seminal fluid, urine, amniotic fluid, cerebrospinal fluid, bronchoalveolar lavage fluid, tears, mucous, tissue, tissue homogenate, cellular extract and spinal fluid. In a specific embodiment, the sample is saliva. In another specific embodiment, the sample is blood. In various embodiments described above, the sample is dispersed in a sample buffer. In one embodiment, the sample buffer comprises a phosphate buffered saline solution.

In various embodiments described above, the system further comprises a conjugation reaction buffer. In one embodiment, the conjugation reaction buffer comprises about 5 mM Potassium Phosphate, about 5 mg/mL PEG20, and wherein the buffer has a pH between about 7.0-7.5. In various embodiments described above, the system further comprises a blocking buffer. In one embodiment, the blocking buffer comprises about 4 mM borate and about 1% BSA.

In one embodiment, the sample pad comprises a porous material. The porous material comprises a matrix selected from the group consisting of a glass fiber, a nitrocellulose, a nitrocellulose blend with polyester, a nitrocellulose blend with cellulose, an untreated paper, a porous paper, and an acrylonitrile copolymer. In one embodiment, the porous material comprises a matrix comprising a glass fiber. In one embodiment, the sample pad is pretreated with a buffered solution. In some embodiments, the sample pad is pretreated with a buffered solution comprising phosphate buffered saline (PBS). In one embodiment, the conjugate pad comprises a matrix comprising a glass fiber. In various embodiments described above, the first and second solid support comprises a gold nanoparticle. In one embodiment, the membrane is a nitrocellulose membrane.

In many embodiments, the conjugate pad is prepared by loading at least about 15 μg/mL of the first anti-coronavirus antibody onto the conjugate pad. In other embodiments, the conjugate pad is prepared by loading at least about 25 μg/mL of the first anti-coronavirus antibody onto the conjugate pad. In some embodiments, the conjugate pad comprises not more than about 10 μL of OD₂₀ first mobilizable conjugate. In some embodiments, the conjugate pad is prepared by loading at least about 15 μg/mL of the first anti-influenzavirus antibody onto the conjugate pad. In other embodiments, the conjugate pad is prepared by loading at least about 25 μg/mL of the first anti-influenzavirus antibody onto the conjugate pad. In some embodiments, the conjugate pad comprises not more than about 10 μL of OD₂₀ second mobilizable conjugate.

In another aspect, the present disclosure provides a kit for the detection of a coronavirus and an influenzavirus, comprising a rapid detection test system as described herein, and reagents and/or instructions for use.

In another related aspect, the present disclosure provides a method for the rapid detection of a coronavirus, or variant thereof, and an influenzavirus, in a biological sample. The method comprises the steps of a) dispersing a biological sample suspected of having either a coronavirus antigen or an influenzavirus antigen, in a sample buffer; b) contacting the biological sample dispersed in the sample buffer with a sample pad comprising a porous material through which the biological sample can flow by capillary action, c) contacting the sample of step b) with a conjugate pad such that the contacting is performed under conditions that permit the capillarity of the first and second mobilizable conjugates formed in the conjugate pad. The conjugate pad comprises a first solid support to which a first anti-coronavirus antibody is attached, forming a first mobilizable conjugate. The first anti-coronavirus antibody of the first mobilizable conjugate is capable of binding to the coronavirus antigen to form a first mobilizable conjugate-antigen complex. The conjugate pad further comprises a second solid support to which a first anti-influenzavirus antibody is attached, forming a second mobilizable conjugate. The first anti-influenzavirus antibody of the second mobilizable conjugate is capable of binding to the influenzavirus antigen to form a second mobilizable conjugate-antigen complex. The method further comprises the steps of contacting the first and the second mobilizable conjugate-antigen complexes with first and second test zones, respectively, on a membrane. The first test zone comprises a second anti-coronavirus antibody immobilized to the membrane. The second anti-coronavirus antibody is capable of binding to the first mobilizable conjugate-antigen complex to form a first immobilizable complex. The first immobilizable complex produces a first detectable signal at the first test zone indicating the presence of the coronavirus antigen in the biological sample. The second test zone comprises a second anti-influenzavirus antibody immobilized to the membrane. The second anti-influenzavirus antibody is capable of binding to the second mobilizable conjugate-antigen complex to form a second immobilizable complex. The second immobilizable complex produces a second detectable signal at the second test zone indicating the presence of the influenzavirus antigen in the biological sample.

In one embodiment of the method, the second anti-coronavirus antibody is conjugated to a detectable agent capable of producing a first detectable signal. In one embodiment of the method, the second anti-influenzavirus antibody is conjugated to a detectable agent capable of producing a second detectable signal.

In one embodiment, the method further comprises at least one control zone downstream of the first and second test zones. The at least one control zone comprises an immunoglobulin immobilized to the membrane. The immunoglobulin is capable of binding to the first anti-coronavirus antibody to form a third immobilizable complex. The immunoglobulin is further capable of binding to the first anti-influenzavirus antibody to form a fourth immobilizable complex.

In one embodiment, the membrane of the method described above comprises a first control zone and a second control zone. The first control zone and the second control zone are downstream from the first test zone and the second test zone, respectively. The first control zone comprises a first immunoglobulin immobilized to the membrane. The first immunoglobulin is capable of binding to the first mobilizable conjugate to form the third immobilizable complex. The second control zone comprises a second immunoglobulin immobilized to the membrane such that the second immunoglobulin is capable of binding to the second mobilizable conjugate, forming the fourth immobilizable complex. The third immobilizable complex produces a third detectable signal at the first control zone indicating capillarity of the biological sample, and the fourth immobilizable complex produce a fourth detectable signal at the second control zone indicating capillarity of the biological sample.

In various embodiments of the method described above, the first and second detectable signals are capable of visual detection without instrumentation. In some embodiments of the method, the first, second, third and fourth detectable signals are capable of visual detection without instrumentation.

In various embodiments of the method, the first anti-coronavirus antibody is capable of binding to at least one coronavirus selected from the group consisting of 229E, NL63, OC43, HKU1, MERS-CoV, SARS-CoV and SARS-CoV-2. In another embodiment, the second anti-coronavirus antibody is capable of binding to at least one coronavirus selected from the group consisting of 229E, NL63, OC43, HKU1, MERS-CoV, SARS-CoV and SARS-CoV-2. In some embodiments, the coronavirus antigen described in the method to detect a coronavirus is selected from the group consisting of a spike protein (S), a receptor-binding (RBD) protein, a S1 protein, a S2 protein, a whole protein (S1+S2), and a nucleocapsid protein (NP). In a specific embodiment, the coronavirus antigen is a nucleocapsid protein. In another specific embodiment, the coronavirus antigen is a spike protein.

In many embodiments of the method, the first anti-influenzavirus antibody is capable of binding to at least one influenzavirus antigen selected from the group viruses consisting of influenzavirus A, influenzavirus B, influenzavirus C, and influenzavirus D. In another embodiment, the second anti-influenzavirus antibody is capable of binding to at least one influenzavirus antigen selected from the group viruses consisting of influenzavirus A, influenzavirus B, influenzavirus C, and influenzavirus D.

In many embodiments of the method, the sample is selected from the group consisting of oral fluid, saliva, blood, blood plasma, blood serum, nasopharyngeal fluid, amniotic fluid, breast milk, vaginal secretions, semen, seminal fluid, urine, amniotic fluid, cerebrospinal fluid, bronchoalveolar lavage fluid, tears, mucous, tissue, tissue homogenate, cellular extract and spinal fluid. In one embodiment, the biological sample is an oral specimen. In a specific embodiment, the biological sample is saliva. In another embodiment, the biological sample is a nasal specimen. In another specific embodiment, the sample is blood. In various embodiments described above, the sample is dispersed in a sample buffer. In one embodiment, the sample buffer comprises a phosphate buffered saline solution.

In various embodiments, method described above further comprises a pretreatment step. The pretreatment step comprises contacting the sample pad with a phosphate buffered saline solution. In various embodiments, method described above further comprises a blocking step. The blocking step comprises contacting the conjugation pad with a blocking buffer for at least 30 minutes prior to step b). In one embodiment, the blocking buffer comprises about 4 mM borate and about 1% BSA.

In many embodiments of the method described above, the conjugate pad is prepared by loading between about 10 μg/mL to 30 μg/mL of the first anti-coronavirus antibody onto the conjugate pad. In one embodiment, the conjugate pad is prepared by loading at least about 15 μg/mL of the first anti-coronavirus antibody onto the conjugate pad. In another embodiment, the conjugate pad is prepared by loading at least about 25 μg/mL of the first anti-coronavirus antibody onto the conjugate pad. In some embodiments, the conjugate pad comprises not more than about 10 μL of OD₂₀ first mobilizable conjugate. In some embodiments, the second anti-coronavirus antibody is immobilized on the membrane at a concentration of between 10 μg/mL to 30 μg/mL. In one embodiment, the second anti-coronavirus antibody is immobilized on the membrane at a concentration of about 15 μg/mL. In another embodiment, the first anti-coronavirus antibody is immobilized on the first solid support at a concentration of about 15 μg/mL and the second anti-coronavirus antibody is immobilized on the membrane at a concentration of about 15 μg/mL.

In various embodiments of the method described above, the conjugate pad is prepared by loading between about 10 μg/mL to 30 μg/mL of the first anti-influenzavirus antibody onto the conjugate pad. In one embodiment, the conjugate pad is prepared by loading at least about 15 μg/mL of the first anti-influenzavirus antibody onto the conjugate pad. In another embodiment, the conjugate pad is prepared by loading at least about 25 μg/mL of the first anti-influenzavirus antibody onto the conjugate pad. In some embodiments, the conjugate pad comprises not more than about 10 μL of OD₂₀ second mobilizable conjugate. In some embodiments, the second anti-influenzavirus antibody is immobilized on the membrane at a concentration of between 10 μg/mL to 30 μg/mL. In one embodiment, the second anti-influenzavirus antibody is immobilized on the membrane at a concentration of about 15 μg/mL. In another embodiment, the first anti-influenzavirus antibody is immobilized on the first second support at a concentration of about 15 μg/mL and the second anti-influenzavirus antibody is immobilized on the membrane at a concentration of about 15 μg/mL.

In various embodiments of the method described above, the membrane is a nitrocellulose membrane.

In another aspect, the present disclosure provides a detection system comprising a lateral flow device, a CRISPR effector system, a detection construct, and a first capture region comprising a first binding agent. In one embodiment, the CRISPR effector system comprises a CRISPR effector protein and one or more guide sequences, each guide sequence configured to bind one or more target molecules.

In one embodiment, the lateral flow device referenced herein comprises a substrate comprising a first end. In some embodiments, the first end comprises a sample loading portion and a first region loaded with a detectable ligand.

In one embodiment, the sample loading portion further comprises one or more amplification reagents to amplify the one or more target molecules. In some embodiments, the reagents to amplify the one or more target molecules comprise nucleic acid sequence-based amplification (NASBA), recombinase polymerase amplification (RPA), loop-mediated isothermal amplification (LAMP), strand displacement amplification (SDA), helicase-dependent amplification (HDA), nicking enzyme amplification reaction (NEAR), PCR, multiple displacement amplification (MDA), rolling circle amplification (RCA), ligase chain reaction (LCR), or ramification amplification method (RAM).

In one embodiment, the detection construct comprises an RNA or DNA oligonucleotide, comprising a first molecule on a first end and a second molecule on a second end. In a specific embodiment, the first molecule is FITC and the second molecule is biotin, or vice versa.

In one embodiment, the first capture region comprises a first binding agent that specifically binds the first molecule of the reporter construct. In a specific embodiment, the first binding agent is an antibody that is fixed or otherwise immobilized to the first capture region. In one embodiment, the antibody is an anti-FITC antibody. In a particular embodiment, the antibody is an anti-biotin antibody.

In one embodiment, the CRISPR effector protein is an RNA-targeting effector protein, a DNA-targeting protein, or a combination thereof. In a specific embodiment, the RNA-targeting effector protein is a Cas12. In one embodiment, the DNA-targeting effector protein is a Cas 12.

In various embodiments of the system, the one or more guide sequences that are diagnostic for a viral infection. In one embodiment, the viral infection is caused by a RNA virus. In one embodiment, the viral infection is caused by a double-stranded RNA virus, a positive sense RNA virus, a negative sense RNA virus, a retrovirus, or a combination thereof. In another embodiment, the viral infection is caused by a Coronaviridae virus, a Picornaviridae virus, a Caliciviridae virus, a Flaviviridae virus, a Togaviridae virus, a Bornaviridae, a Filoviridae, a Paramyxoviridae, a Pneumoviridae, a Rhabdoviridae, an Arenaviridae, a Bunyaviridae, an Orthomyxoviridae, or a Deltavirus. In yet another embodiment, the viral infection is caused by SARS-COV-2 Coronavirus, SARS, Poliovirus, Rhinovirus, Hepatitis A, Norwalk virus, Yellow fever virus, West Nile virus, Hepatitis C virus, Dengue fever virus, Zika virus, Rubella virus, Ross River virus, Sindbis virus, Chikungunya virus, Borna disease virus, Ebola virus, Marburg virus, Measles virus, Mumps virus, Nipah virus, Hendra virus, Newcastle disease virus, Human respiratory syncytial virus, Rabies virus, Lassa virus, Hantavirus, Crimean-Congo hemorrhagic fever virus, Influenza, or Hepatitis D virus.

In one embodiment, the first detection construct comprises Tye665 as a first molecule and Alexa-fluor-488 as a second molecule or vice versa. In a particular embodiment, the CRISPR effector protein is an RNA-targeting or a DNA-targeting effector protein.

### BRIEF DESCRIPTION OF THE FIGURES

FIG. 1 shows a schematic overview of certain test components of a sandwich-format lateral flow immunoassay. The assay components include a sample pad, a conjugate pad, a nitrocellulose membrane, and a wick pad.

FIG. 2 shows a schematic overview of how the sandwich-format lateral flow immunoassay works. In the presence of nucleocapsid protein, the conjugate will bind to the nucleocapsid protein and travel downstream toward the nitrocellulose membrane. Upon interaction with the test line capture reagent, the conjugate will be immobilized at the test line region and create a uniform, visible signal indicating positive result. If no nucleocapsid protein is present, the conjugate will continue to travel up the test strip without binding to the test line. Excess conjugate will always bind to the control line region to indicate the test was performed correctly.

FIGS. 3A-B show the test strip results of experiments to evaluate affinity reagents with contrived samples. Nucleocapsid protein was diluted in 10 mM PBS between 1 ng/mL to 100 μg/mL and evaluated with two sandwich assay formats. FIG. 3A shows test strips of the sandwich assay prepared with Conjugate Antibody B (Clone B3451M) and Test Line Antibody A (Clone B3449M). FIG. 3B shows test strips of the sandwich assay prepared with Conjugate Antibody A (Clone B3449M) and Test Line Antibody B (Clone B3451M). The results demonstrate that the sandwich assay prepared with Conjugate Antibody B and Test Line Antibody A elicited strong signal at 100 μg/mL. The reverse sandwich format in FIG. 3B failed to elicit signal at any concentrations evaluated.

FIG. 4 shows the MDI 10 test strip results of the evaluation of Antibody C conjugates. Testing was performed using nucleocaspid controls for the (NP) Swab SARS-CoV-2 Antigen Test. In the figure, A=15 μg/mL Antibody Loading; B=25 μg/mL Antibody Loading, and 1=15-minute incubation; 2=30-minute incubation; and 3=60-minute incubation. Faint non-specific binding (NSB) was observed with negative testing. Moderate-to-strong test line signal intensity was observed with all medium positive tests. The intensity from one condition to another is not easily modulated, suggesting the testing efforts employed no significant impact on assay performance.

FIG. 5 shows test strip results of the evaluation of sample pad types and pretreatment with spiked saliva. Testing was performed with saliva prepared to antigen concentrations of 0 μg/mL, 0.0001 μg/mL, and 0.001 μg/mL, respectively. Testing on strips with pretreated 8951 sample pads exhibited higher levels of non-specific binding with the negative sample (0 μg/mL). Test line signal at 0.001 μg/mL was strong for both sample pad variants. Test line signal at 0.0001 μg/mL was observed at slightly stronger intensity than the NSB observed with the negative sample on 8950 sample pad test strips, suggesting there may be sensitivity at sample concentrations as low as 0.0001 μg/mL (100 picogram/mL) if the non-specific signal is removed from the test.

### DETAILED DESCRIPTION OF THE INVENTION

The compositions and methods as described herein are for the rapid detection of coronavirus, and in particular, SARS-CoV-2 antigens in a biological sample. The present disclosure also provides compositions and methods for the rapid detection of coronavirus (e.g., SARS-CoV-2) and influenzavirus, in a single test system.

SARS-CoV-2 (a.k.a. Coronavirus Disease 2019, covid-19 virus, 2019-nCoV, severe acute respiratory syndrome coronavirus 2, Wuhan-hu-1, or COVID-19 virus) is a virus that first emerged in China in late 2019. SARS-CoV-2 refers to a virus that belongs to a family of viruses, the Coronaviridae, a group IV ((+) ssRNA) virus of the genus betacoronavirus following the nomenclature of the Coronavirus Study group (de Groot 2013). Generally, coronaviruses are zoonotic, which means that the viruses are transmitted between animals and people. SARS-CoV-2 was isolated from respiratory epithelium of patients and first described in 2020 by Xu et a (Viruses 2020, 12, 244; doi:10.3390/v12020244). The complete genome of the human SARS-CoV-2 virus has been deposited under the GenBank accession number MN908947.3. In addition, the World Health Organization (WHO) maintains a website (see the World Wide Web at.who.int/health-topics/coronavirus) reporting on the newest developments and information regarding this virus.

Coronaviruses, a genus in the family Coronaviridae, are large, enveloped RNA viruses that cause highly prevalent diseases in humans and domestic animals. Coronavirus particles are irregularly-shaped, 60-220 nm in diameter, with an outer envelope bearing distinctive “crown-like” appearance that gives the family its name. There are four main sub-groupings of coronaviruses, known as alpha, beta, gamma, and delta. Coronaviruses have the largest genomes of all RNA viruses and replicate by a unique mechanism which results in a high frequency of recombination. Virions mature by budding at intracellular membranes and infection with some coronaviruses induces cell fusion. The virion of the coronaviruses typically contains several viral structural proteins including the ^{˜}140 kDa spike glycoprotein (S), a 23 kDa membrane glycoprotein (M) and a ^{˜}10 kDa protein (E).

Human coronaviruses were first identified in the mid-1960s with certain more prominent viruses, including, MERS-CoV (the beta coronavirus that causes Middle East Respiratory Syndrome, or MERS), SARS-CoV (the beta coronavirus that causes severe acute respiratory syndrome, or SARS) and SARS-CoV-2 (the novel coronavirus that causes coronavirus disease 2019-2020, or COVID-19). Most human coronaviruses (HcoVs) do not grow in cultured cells, therefore relatively little is known about them. Viral entry is hypothesized to occur via endocytosis and membrane fusion (probably mediated by S) and replication likely occurs in the cytoplasm.

Coronaviruses infect a variety of mammals and birds. The exact number of human isolates may not be known as many may not be sufficiently capable of being grown in culture. In some humans, coronaviruses cause, inter alia, respiratory infections (common), including Severe Acute Respiratory Syndrome (SARS), and enteric infections.

Coronaviruses are in some respects transmitted by aerosols of respiratory secretions, by the fecal-oral route, and by mechanical transmission. Most coronavirus growth occurs in epithelial cells. Occasionally the liver, kidneys, heart or eyes may be infected, as well as other cell types such as macrophages. In cold-type respiratory infections, growth appears to be localized to the epithelium of the upper respiratory tract, but there is currently no adequate animal model for the human respiratory coronaviruses. Clinically, most infections cause a mild, self-limited disease (classical “cold” or upset stomach), but there may be in some infections neurological complications. The novel SARS-CoV-2 virus appears to localize to the pulmonary cells of the lower respiratory tract, causing severe respiratory complications leading to death in select patients and patient populations. In some patients, the SARS-CoV-2 virus results in SARS (Severe Acute Respiratory Syndrome), a recognized type of viral pneumonia, with symptoms including fever, a dry cough, dyspnea (shortness of breath), headache, and hypoxemia (low blood oxygen concentration). Typical laboratory findings include lymphopenia (reduced lymphocyte numbers) and mildly elevated aminotransferase levels (indicating liver damage). Death may result from progressive respiratory failure due to alveolar damage.

The S protein of coronavirus is an important determinant of tissue tropism, as it binds to cellular receptors on the host cell and it is also crucial for virus and cellular membrane fusion. A “structural protein” as used herein refers to a protein of the coronavirus, such as those encoded by the S, E, M and N genes, as well as any other structural proteins now known or later identified in the coronavirus and in particular in the SARS-CoV-2 virus genome.

The present disclosure also provides compositions and methods for the rapid detection of coronavirus (e.g., SARS-CoV-2) and influenzavirus, in a single detection system. Influenza viruses belong to the genus Orthomyxoviridae, a genus of RNA viruses that includes the Influenzavirus A, Influenzavirus B, Influenzavirus C, Influenzavirus D, viruses that cause influenza in vertebrates. There are two main types of influenza (flu), the influenza A and B viruses, that routinely spread in people (human influenza viruses) and are typically responsible for seasonal flu epidemics each year.

The influenza A virus is an enveloped, negative-strand RNA virus. The genome of influenza A virus is contained on eight single (non-paired) RNA strands the complements of which code for eleven proteins (HA, NA, NP, M1, M2, NS1, NEP, PA, PB1, PB1-F2, PB2). The total genome size is about 14,000 bases. The segmented nature of the genome allows for the exchange of entire genes between different viral strains during cellular cohabitation. The eight RNA segments are as follows. 1) HA encodes hemagglutinin (about 500 molecules of hemagglutinin are needed to make one virion); 2) NA encodes neuraminidase (about 100 molecules of neuraminidase are needed to make one virion); 3) NP encodes nucleoprotein; 4) M encodes two proteins (the M1 and the M2) by using different reading frames from the same RNA segment (about 3000 M1 molecules are needed to make one virion); 5) NS encodes two proteins (NS1 and NEP) by using different reading frames from the same RNA segment; 6) PA encodes an RNA polymerase; 7) PB1 encodes an RNA polymerase and PB1-F2 protein (induces apoptosis) by using different reading frames from the same RNA segment; 8) PB2 encodes an RNA polymerase.

There are several subtypes of influenza A, named according to an H number (for the type of hemagglutinin) and an N number (for the type of neuraminidase). Currently, there are 16 different H antigens known (HI to HI6) and nine different N antigens known (N1 to N9). Each virus subtype has mutated into a variety of strains with differing pathogenic profiles; some pathogenic to one species but not others, some pathogenic to multiple species. Exemplary Influenza A virus subtypes that have been confirmed in humans, include, but are not limited to H1N1 which caused the “Spanish Flu” and the 2009 swine flu outbreak; H2N2 which caused the “Asian Flu” in the late 1950s; H3N2 which caused the Hong Kong Flu in the late 1960s; H5N1, considered a global influenza pandemic threat through its spread in the mid-2000s; H7N7; H1N2 which is currently endemic in humans and pigs; and H9N2, H7N2, H7N3, H5N2, H10N7. Some influenza A variants are identified and named according to the known isolate to which they are most similar, and thus are presumed to share lineage (e.g., Fujian flu virus-like); according to their typical host (example Human flu virus); according to their subtype (example H3N2); and according to their pathogenicity (example LP, Low Pathogenic). Thus, a flu from a virus similar to the isolate A/Fujian/411/2002(H3N2) can be called Fujian flu, human flu, and H3N2 flu.

Influenza B virus is almost exclusively a human pathogen, and is less common than influenza A. Influenza B virus mutates at a rate 2-3 times lower than type A and consequently is less genetically diverse, with only one influenza B serotype. As a result of this lack of antigenic diversity, a degree of immunity to influenza B is usually acquired at an early age. However, influenza B mutates enough that lasting immunity is not possible. This reduced rate of antigenic change, combined with its limited host range (inhibiting cross species antigenic shift), ensures that pandemics of influenza B do not occur.

### Definitions

The term “about”, as used herein, refers to a value that is within 10% above or below the value being described.

The term “control zone” or “control line”, used interchangeably herein, refers to a region of a membrane (e.g., a nitrocellulose membrane) that contains at least one control binding agent (e.g., an immunoglobulin) immobilized to the membrane, which is capable of binding a mobilizable conjugate.

The term “immobilizable conjugate”, as used herein, refers to a complex formed between a capture agent (e.g., an anti-SARS-CoV-2 antibody or an anti-influenza virus antibody) and a solid support (e.g., a gold nanoparticle), which is not capable of substantially unidirectional flow via capillary action.

The term “immobilizable conjugate-antigen complex”, as used herein, refers to a complex formed between a mobilizable conjugate and an analyte (e.g., a SARS-CoV-2 antigen or an influenza virus antigen), which is not capable of substantially unidirectional flow via capillary action.

The term “lateral flow”, as used herein, refers to a substantially unidirectional flow of a mobile phase (e.g., a biological sample dispersed in a buffer) by capillary action.

The term “matrix”, as used herein, refers to any porous material capable of facilitating lateral flow.

The term “mobilizable conjugate”, as used herein, refers to a complex formed between a capture agent (e.g., an anti-SARS-CoV-2 antibody or an anti-influenza virus antibody) and a solid support (e.g., a gold nanoparticle), which is capable of substantially unidirectional flow via capillary action.

The term “mobilizable conjugate-antigen complex”, as used herein, refers to a complex formed between a mobilizable conjugate and an analyte (e.g., a SARS-CoV-2 antigen or an influenza virus antigen), which is capable of substantially unidirectional flow via capillary action.

The term “sample”, as used herein, refers to any medium (e.g., a fluid) suspected of containing an analyte (e.g., an antigen) of interest (e.g., a SARS-CoV-2 antigen).

The term “sample loading”, as used herein, refers to the application of a sample (e.g., a biological sample) to a sample pad such that the sample is capable of substantially unidirectional flow along a solid support via capillary action toward a test zone(s) and/or a control zone(s) of a membrane.

The term “substantially”, as used herein, refers to a quantitative state that indicates a complete or near complete degree of a feature or characteristic of interest.

The term “test strip”, as used herein, refers to a porous membrane capable of facilitating substantially unidirectional flow along a solid support via capillary action of a sample containing an analyte(s) of interest (e.g., a SARS-CoV-2 antigen). A test strip, as described herein, includes, but is not limited to, one or more sample pads, one or more conjugate pads, one or more porous membrane(s), one or more filters and/or backing materials (e.g., plastic or cardboard backing materials) required to perform the invention described herein.

The term “test zone” or “test line”, used interchangeably herein, refers to a region of a membrane (e.g., a nitrocellulose membrane) that contains at least one analyte binding agent (e.g., an anti-SARS-CoV-2 antibody) immobilized to a membrane, which is capable of binding an analyte (e.g., a SARS-CoV-2 antigen).

### I. Rapid Detection Systems

Various aspects of the present disclosure are directed to immunoassays that utilize specific binding moieties and capture moieties for the qualitative and/or quantitative analysis of one or more analytes (e.g., antigens) of interest in a sample (e.g., a biological sample). The compositions provided herein are useful in a variety of assays that are utilized to detect one or more analytes (e.g., antigens) that are determinants of an infection. The compositions described herein can be incorporated into one or more assays including, but not limited to, an immunoassay, a sandwich immunoassay and a blocking assay. As an example, the detection systems provided herein can be part of a lateral flow sandwich type immunoassay to detect analytes (e.g., antigens) of interest (e.g., a SARS-CoV-2 antigen, an influenza virus antigen) in the sample.

### A. SARS-CoV-2 Rapid Detection System

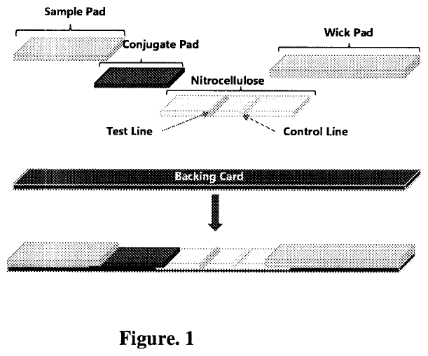

The present disclosure provides compositions for rapidly detecting SARS-CoV-2 in a sample (e.g., an oral sample, a mucus sample, a nasal sample, a sputum sample, a blood sample). In one aspect, the present disclosure provides a rapid detection system for the detection of SARS-CoV-2, or a variant thereof. In some embodiments, the system comprises a sample pad, a conjugate pad and a membrane. Although, in certain embodiments, the components of the system may be placed in various arrangements according to the assay format intended and the type of assay to be performed, generally, the components of the rapid detection system for the detection of SARS-CoV-2, or a variant thereof provided herein are arranged to facilitate the unidirectional flow of the sample from the sample pad, to the conjugate pad, and subsequently to the membrane pad. In certain embodiments, the sample pad comprises a porous material. In some embodiments, the porous material comprises a matrix selected from the group consisting of a glass fiber, a nitrocellulose, a nitrocellulose blend with polyester, a nitrocellulose blend with cellulose, an untreated paper, a porous paper, and an acrylonitrile copolymer. Common porous membranes include, but are not limited to, fiberglass, porous nitrocellulose or polyethylene. In some embodiments, the porous material comprises a matrix comprising a glass fiber. In particular embodiments, the sample pad comprises a backing pad for structural support as shown in the schematic of an exemplary rapid detection system for the detection of SARS-CoV-2 (FIG. 1). In some embodiments described herein, the sample pad, the conjugate pad and the membrane of the rapid detection system are present in a single test strip.

In one embodiment described herein, a sample (e.g., a biological sample) is loaded onto a sample pad. In certain embodiments, a sample may be obtained from a mammal (e.g., a human, a non-human primate, a dog, a cat, a horse, a pig, etc.) and selected from the group consisting of an oral sample, saliva, blood, blood plasma, blood serum, nasopharyngeal fluid, amniotic fluid, breast milk, vaginal secretions, semen, seminal fluid, urine, amniotic fluid, cerebrospinal fluid, nasal fluid, bronchoalveolar lavage fluid, tears, mucous, tissue, tissue homogenate, cellular extract, spinal fluid, fecal specimen and any combination of two or more of the foregoing. In yet other embodiments, the sample is a human physiological fluid, a cell culture, or an environmental sample(s). In some embodiments, the sample is taken from a human using a swab (e.g., a nasal swab or a throat swab). In a specific embodiment, the sample is saliva. In another embodiment, the sample is blood. In the embodiments where the sample is blood, the porous material of the sample can also act as a filter to filter out (e.g., retain) red blood cells and/or white blood cells, but allow the serum or plasma proceed through the mobile phase. In certain embodiments described herein, the porous material of the sample pad comprises a matrix. In certain other embodiments, upon loading of a sample onto a sample pad, the sample proceeds to move via lateral flow without substantial preferential retention of any one or more of the components of the sample as would occur with certain other media e.g., the stationary phase of a gel filtration column composed of Sepharose beads) capable of substantially adsorbing or altering the flow of one or more components. For example, upon loading of a sample onto a sample pad, dispersed components of a sample (e.g., a SARS-CoV-2 antigen and/or an influenza virus antigen in a sample dispersed in a sample buffer) proceed to move by capillary action at substantially equal rates and with relatively unimpaired flow laterally through a matrix. Non-limiting examples of a matrix described herein include nitrocellulose, nitrocellulose blends (including those selected from the group consisting of a glass fiber, a nitrocellulose, a nitrocellulose blend with polyester, a nitrocellulose blend with cellulose), an untreated paper, a porous paper, and an acrylonitrile copolymer. Other porous materials useful for the instant disclosure include those that allow lateral flow. Typically, the matrix will be in the form of a strip through which a sample may flows laterally (horizontally) although the matrix could be set up in layers through which the test fluid could flow vertically from top to bottom or vice versa.

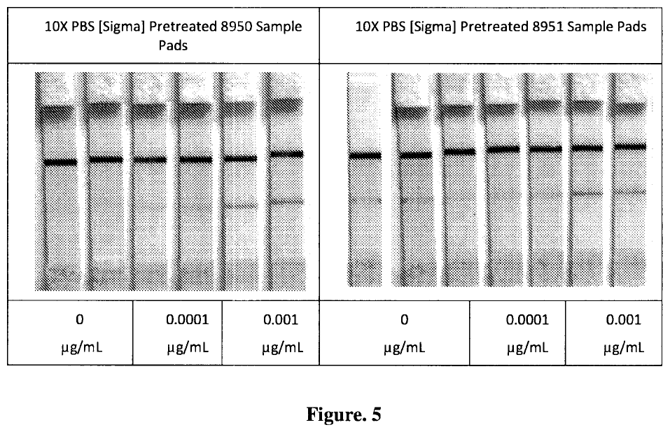

In certain embodiments described herein, the flow of the sample through the rapid detection system is facilitated by the addition of a buffer. In the various embodiments described herein, the sample is dissolved, dispersed, suspended and/or reconstituted in one or more appropriate buffers. In some embodiments, the sample is dispersed in a buffer, referred to as the “sample buffer” prior to loading onto a sample pad. In other embodiments, the sample is loaded directly onto a sample pad that has been pre-treated with the sample buffer. In certain embodiments described herein, the sample buffer comprises a phosphate buffered saline solution. In certain other embodiments, an Ahlstrom grade 8950 and/or a grade 8951 glass fiber sample pad are pretreated with 10× phosphate buffered saline (PBS) solutions. In some embodiments, 10×PBS solutions are prepared from packets acquired through Sigma Aldrich or Thermo Fisher and applied to a sample pad as described herein. In some embodiments, an Ahlstrom 8950 sample pad pretreated with 10×PBS exhibited less non-specific binding compared to a sample pad prepared using 8951 sample pads. In some embodiments described herein, the pretreated sample pads improve the test performance, allowing signal modulation between negative samples and samples spiked to 0.0001 μg/mL nucleocapsid protein (FIG. 5).

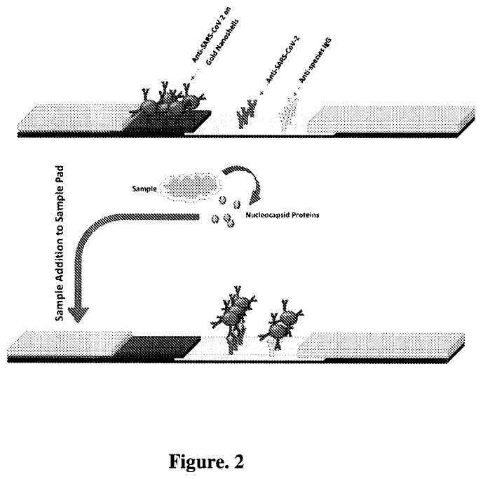

In some embodiments, a conjugate pad is composed of a hydrophobic material, such as glass fiber (e.g., Ahlstrom 8950) and contains a capture agent that can react with an analyte (e.g., an antigen) in a sample (e.g., a biological sample). In various embodiments described herein, the conjugate pad is coupled to the sample pad and is in a flow path that is downstream from the sample pad. In certain embodiments, the conjugation pad includes a capture agent attached to a solid support. In some embodiments, with capture agent is capable of specifically binding to the analyte (e.g., antigen) of interest. In other embodiments, the capture agent is an antibody, or a fragment thereof. In another embodiment, the capture agent is a monoclonal antibody. In another embodiment, the capture agent is a polyclonal antibody. In another embodiment, the conjugation pad includes a solid support, wherein a first anti-SARS-CoV-2 antibody is attached to the solid support to form a mobilizable conjugate, such as that illustrated in the schematic in FIG. 2. In some embodiments, the first anti-SARS-CoV-2 antibody of the mobilizable conjugate is capable of binding to a SARS-CoV-2 antigen to form a mobilizable conjugate-antigen complex. In some embodiments, an antigen includes, without limitation, a protein, a viral particle, a viral subunit, a chemical compound, a polypeptide, a carbohydrate, a nucleic acid, a lipid, and a glycoprotein that may be characteristic of the subject's condition. In some embodiments, the antigen is a SARS-CoV-2 antigen. In other embodiments, the SARS-CoV-2 antigen is selected from the group consisting of a spike protein (S protein), a receptor-binding (RBD) protein, a S1 protein, a S2 protein, a whole protein (S1+S2), and a nucleocapsid protein (NP). In a specific embodiment, the SARS-CoV-2 antigen is a nucleocapsid protein. In another embodiment, the SARS-CoV-2 antigen is a spike protein.

In some embodiments, the anti-SARS-CoV-2 antibody is attached to a solid support to form a mobilizable conjugate. In certain embodiments, the mobilizable conjugate formed between a capture agent (e.g., the first anti-SARS-CoV-2 antibody) and a solid support (e.g., a gold nanoparticle) is mediated by covalent interactions. In other embodiments, the mobilizable conjugate formed between a capture agent (e.g., the first anti-SARS-CoV-2 antibody) and a solid support (e.g., a gold nanoparticle) is mediated by non-covalent interactions, e.g., hydrophobic and/or ionic interactions. The solid support can be any material that provides a surface or a point of attachment for the capture agent and/or includes a detectable material including, but not limited to, a colored material, a fluorescent material, and a chemiluminescent material. In one embodiment, the anti-SARS-CoV-2 antibody is attached to a solid support that includes a colloidal gold particle. In other embodiment, the anti-SARS-CoV-2 antibody is attached to a solid support that includes a gold nanoparticle. In other embodiments, the solid support includes a protein, such as biotin or strepavidin. In some embodiments, an anti-SARS-CoV-2 nucleocapsid protein IgG is attached to the surface of a gold nanoparticle to form a mobilizable conjugate. In a specific embodiment, the anti-SARS-CoV-2 nucleocapsid protein IgG is the anti-SARS-CoV-2 nucleocapsid protein IgG from Arista Biologicals. In other embodiments, a first anti-SARS-CoV-2 Spike protein IgG is attached to the surface of a gold nanoparticle to form a mobilizable conjugate. In various embodiments described herein, the mobilizable conjugate is diffusibly bound to a matrix in a conjugate pad, but capable of dispersing (e.g., by capillary action) with the mobile phase and/or is capable of substantially unidirectional flow via a mobile phase by capillary action. In some embodiments, the anti-SARS-CoV-2 antibody of the mobilizable conjugate is capable of binding to a SARS-CoV-2 antigen to form a mobilizable conjugate-antigen complex.

In some embodiments described herein, the conjugate is loaded onto to a conjugate pad and is subsequently dried. In other embodiments, a conjugation reaction is performed by mixing a lyophilized form of a capture agent with a dry form of the solid support. In another embodiment, a lyophilized powder of an anti-SARS-CoV-2 nucleocapsid protein IgG is mixed with dry form of a gold nanoparticles to form a mobilizable conjugate. In another embodiment, a lyophilized powder of an anti-SARS-CoV-2 Spike protein IgG is mixed with a dry form of a gold nanoparticle to form a mobilizable conjugate. In various embodiments described herein, the conjugation reaction to form the mobilizable conjugate comprises a conjugation reaction buffer. In a particular embodiment, a conjugation reaction buffer includes about 5 mM Potassium Phosphate and about 5 mg/mL PEG20, wherein the buffer has a pH between about 7.0-7.5. In some embodiments, a conjugation reaction buffer includes about 5 mM Potassium Phosphate, about 5 mg/mL PEG20, at a pH between about 7.0-7.5 is applied to the conjugate pad to rehydrate the mobilizable conjugate. In various embodiments described herein, the conjugate pads can be preblocked with a buffer solution containing a blocking buffer. In some embodiments, the purpose for preblocking the conjugate pad with a blocking buffer solution is to stabilize the conjugate when dried on the conjugate pad. In one embodiment, the blocking buffer comprises about 4 mM borate and about 1% BSA.

In various embodiments described herein, a conjugate pad is prepared by loading at least about 10 μg/mL, at least about 15 μg/mL, at least about 20 μg/mL, at least about 25 μg/mL, at least about 30 μg/mL, or at least about 35 μg/mL of an anti-SARS-CoV-2 antibody onto the conjugate pad. In one embodiment, a conjugate pad is prepared by loading at least about 15 μg/mL of an anti-SARS-CoV-2 antibody onto the conjugate pad. In another embodiment, a conjugate pad is prepared by loading at least about 25 μg/mL of an anti-SARS-CoV-2 antibody onto the conjugate pad. In certain embodiments described herein, a conjugate pad comprises not more than about 1 μL, about 2 μL, about 5 μL, about 10 μL, or about 20 μL, of OD₂₀ mobilizable conjugate. In one embodiment, the conjugate pad comprises not more than about 10 μL of OD₂₀ mobilizable conjugate. In another embodiment, the conjugate pad comprises not more than about 5 μL of OD₂₀ mobilizable conjugate. In various embodiments described herein, a conjugation reaction is performed to facilitate the formation of the mobilizable conjugate comprising an antibody and a gold nanoparticle using 1-ethyl-3-(3-dimethylaminopropyl) carbodiimide/N-Hydroxysuccinimide (EDC/NHS) activation chemistry. In the EDC/NHS activation chemistry used for covalent conjugation of the antibody with the gold particle, EDC is used to activate the carboxyl group on the surface of nanoparticles to create a crosslinker. The resulting intermediate can bind to primary amines on the antibody, but may be unstable and susceptible to hydrolysis. Sulfo-NHS is added with EDC to create a more stable amine-reactive intermediate, which will bind to the primary amines on the antibody. In certain embodiments described herein, a conjugation reaction comprises an EDC loading with not more than about 8 μL, about 10 μL, about 16 μL, about 20 μL, or about 32 μL, of 1 ml OD₂₀ EDC solution. In one embodiment, a conjugation reaction comprises an EDC loading with about 8 L of 1 ml OD₂₀ EDC solution. In another embodiment described herein, a conjugation reaction comprises an NHS loading with not more than about 8 μL, about 16 μL, about 24 μL, about 32 μL, or about 64 μL, of 1 ml OD₂₀ NHS solution. In one embodiment, a conjugation reaction comprises an NHS loading with about 16 μL of 1 ml OD₂₀ NHS solution. In various embodiments described herein, a conjugate pad is prepared by loading the antibody with at least a 15-minute, at least a 30-minute or at least a 60-minute antibody incubation followed by, at least in some embodiments, at least a 15-minute, at least a 30-minute or at least a 60-minute, antibody blocking incubation step. In one embodiment, a conjugate pad is prepared by loading the antibody with at least a 30-minute antibody incubation followed by at least a 30-minute antibody blocking incubation step. In some embodiments, the first anti-SARS-CoV-2 antibody is Antibody B (Clone B3451M). In another embodiment, the first anti-SARS-CoV-2 antibody is an anti-SARS-CoV-2 (ARISTA Biologicals.)

In certain embodiments of the rapid detection systems described herein, mobilizable components (e.g., a mobilizable conjugate and a mobilizable conjugate-antigen complex) migrate through the system (e.g., through the conjugate pad and through the membrane) as described herein. For example, in some embodiments, upon contacting the conjugate pad, the mobile phase (e.g., a buffered solution comprising a sample) hydrates, suspends and/or mobilizes the mobilizable conjugate to proceed through the mobile phase by capillary action downstream through the system toward the membrane (e.g., a nitrocellulose membrane). In some embodiments, the mobilizable conjugate, upon the movement through the system by lateral flow, contacts an antigen (e.g., a SARS-CoV-2 antigen) to form a complex between the mobilizable conjugate and the antigen, referred to herein as a mobilizable conjugate-antigen complex.

In various embodiments of the rapid detections systems described herein, the systems include a membrane comprising a test zone. In some embodiments, the mobilizable conjugate-antigen complex subsequently proceeds (e.g., by lateral flow) through the system from a conjugate pad into a membrane comprising a test zone. In some embodiments, the analyte binding agent is immobilized in a region of the membrane such that the analyte binding agent is capable of binding to the mobilizable conjugate-antigen complex to form a first immobilizable complex at the test line. In preferred embodiments, the formation of the first immobilizable complex produces an observable or detectable signal at the test line indicating the presence of (and, in some embodiments, the amount of) analyte (e.g., a SARS-CoV-2 antigen) in the sample (e.g., a biological sample). In some embodiments, the analyte binding agent is immobilized to the test line through covalent or ionic means by techniques known by those skilled in the art. In certain embodiments, if an analyte of interest (e.g., a nucleocapsid protein of SARS-CoV-2) is present in the sample, the analyte of interest will bind to a cognate anti-SARS-CoV-2 antibody from the mobilizable conjugate to form a mobilizable conjugate-antigen complex. In such embodiments, the mobilizable conjugate-antigen complex will be immobilized at the test line, resulting in a detectable or observable signal indicating positive result. In other embodiments, if no analyte of interest (e.g., a nucleocapsid protein of SARS-CoV-2) is present in the sample, the mobilizable conjugate will continue to flow downstream from the test line, without immobilizing to the test line. Accordingly, in some embodiments, the rapid detection system described herein is capable of detecting SARS-CoV-2, or a variant thereof, by the appearance of a detectable or observable line in the test zone on the membrane. In one embodiment, the second anti-SARS-CoV-2 antibody is Antibody A (Clone B3449M). In another embodiment, the first anti-SARS-CoV-2 antibody is Antibody B (Clone B3451M) and the second anti-SARS-CoV-2 antibody is Antibody A (Clone B3449M). In other embodiments, the “test zone” may be comprised of more than one capture zone for capturing more than one analyte in the sample, in which event, more than one analyte binding agents (e.g., a second anti-SARS-CoV-2 antibody and a second anti-influenza antibody) may be used. For example, the rapid detection systems described herein can simultaneously detect a coronavirus (e.g., SARS-CoV-2) and an influenza virus, while in other embodiments, the rapid detection systems described herein can simultaneously detect two different coronaviruses (e.g., SARS-CoV-2 and MERS-CoV).

In various embodiments of the rapid detections systems described herein, the systems include a membrane comprising one or more control zones, which contain control agents (e.g., an immunoglobulin) immobilized a region of the membrane spatially distinct and, in preferred embodiments, downstream from a test zone. The control agents specifically bind to control binding agents (e.g., a mobilizable conjugate) to form a control binding pair (e.g., a second immobilizable complex). In one embodiment, the “control agent” is an anti-human or anti-mouse IgG antibody, or antibody portion thereof, that is capable of binding to the first anti-SARS-CoV-2 of the mobilizable conjugate to form a second immobilizable complex. The formation of the second immobilizable complex at the control line, in preferred embodiments, produces an observable or detectable signal, indicating capillarity of the sample (i.e., indicating that the sample has permeated or flowed through the system matrix as designed). Excess mobilizable conjugate will bind to the control agent immobilized on the control line to indicate the test was performed correctly, as shown in the exemplary embodiment illustrated in FIG. 2, wherein the liquid front encounters the control line. In some embodiments, the control line is comprised of mouse or human immunoglobulin (IgG) and enables detection of non-specific binding of the mobilizable conjugates to the immobilized immunoglobulin at the control line, thus approximating the level of non-specific binding that will occur at the upstream test line(s). In some embodiments, the signal generated at the control line is used to ensure that high non-specific binding at the analyte-specific test line does not lead to false positive results. In some embodiments, the rapid detections systems described herein can include two control lines, although the use of two control lines is not required. In some embodiments, the two control lines may be composed of the same or different immobilized control agent(s). In other embodiments, one of the control lines can be designated as a high control (“HC”) and the other control line can be designated as a low control (“LC”). The ratio of HC to LC is typically predetermined as one of the internal quality controls when two controls are used.

In some embodiments, the membrane comprises a test zone and a control zone wherein the membrane is selected from the group consisting of nitrocellulose, cellulose acetate, cellulose nitrate, polyamide, polycarbonate, glass fiber, membrane, polyethersulfone, regenerated cellulose (RC), polytetra-fluorethylene, (PTFE), polyester (e.g. Polyethylene Terephthalate), polycarbonate (e.g., 4,4-hydroxy-diphenyl-2,2′-propane), Aluminum Oxide, mixed cellulose ester (e.g., mixture of cellulose acetate and cellulose nitrate), nylon (e.g., Polyamide, Hexamethylene-diamine, and Nylon 66), polypropylene, PVDF, and High Density Polyethylene (HDPE)+nucleating agent “aluminum dibenzoate” (DBS), or a combination thereof. In one embodiment, the membrane is a nitrocellulose membrane. In another embodiment, the nitrocellulose membrane is a MDI10μ-CNPF membrane. In another embodiment, the nitrocellulose membrane is a MDI70-CNPH membrane. In various embodiments described herein, the test lines are striped at least about 0.04 μL/mm, at least about 0.08 μL/mm, at least about 0.12 μL/mm with an antibody concentration of about 0.1 mg/mL, about 0.5 mg/mL, about 1.0 mg/mL or about 2.0 mg/mL.

### B. Coronavirus Rapid Detection System

The present disclosure provides compositions for rapidly detecting a coronavirus, or a variant thereof in a sample (e.g., an oral sample, a mucus sample, a nasal sample, a sputum sample). In one aspect, the present disclosure provides a rapid detection system for the detection of a coronavirus, or a variant thereof. In some embodiments, the system comprises a sample pad, a conjugate pad and a membrane. Although, in certain embodiments, the components of the system may be placed in various arrangements according to the assay format intended and the type of assay to be performed, generally, the components of the rapid detection system for the detection of a coronavirus, or a variant thereof provided herein, are arranged to facilitate the unidirectional flow of the sample from the sample pad, to the conjugate pad, and to the membrane pad. In certain embodiments, the sample pad comprises a porous material. In some embodiments, the porous material comprises a matrix selected from the group consisting of a glass fiber, a nitrocellulose, a nitrocellulose blend with polyester, a nitrocellulose blend with cellulose, an untreated paper, a porous paper, and an acrylonitrile copolymer. In some embodiments, the porous material comprises a matrix comprising a glass fiber. In particular embodiments, the sample pad, and/or the conjugate pad, and/or the membrane, comprises a backing pad for structural support. In some embodiments described herein, the sample pad, the conjugate pad and the membrane of the rapid detection system are present in a single test strip. Common porous membranes include, but are not limited to, fiberglass, porous nitrocellulose or polyethylene. In some embodiments, a sample (e.g., a biological sample) may be obtained from a mammal (e.g., a human, a non-human primate, a dog, a cat, a horse, a pig, etc.) and selected from the group consisting of oral fluid, saliva, blood, blood plasma, blood serum, nasopharyngeal fluid, amniotic fluid, breast milk, vaginal secretions, semen, seminal fluid, fecal specimen, urine, amniotic fluid, cerebrospinal fluid, bronchoalveolar lavage fluid, tears, mucous, tissue, tissue homogenate, cellular extract, spinal fluid, and any combination of two or more of the foregoing. In yet other embodiments, the sample is a human physiological fluid, a cell culture, and an environmental sample(s). In some embodiments, the sample is taken from a human using a swab (e.g., a nasal swab or a throat swab). In a specific embodiment, the sample is saliva. In another embodiment, the sample is blood.

In some embodiments, a conjugate pad is composed of a hydrophobic material, such as glass fiber (e.g., Ahlstrom 8950) and comprises a first anti-coronavirus antibody attached to a first solid support to form a mobilizable conjugate. In some embodiments, the first anti-coronavirus antibody is capable of binding to a coronavirus antigen to form a mobilizable conjugate-antigen complex. In some embodiments, the coronavirus antigen includes, without limitation, a protein, a viral particle, a viral subunit, a chemical compound, a polypeptide, a carbohydrate, a nucleic acid, a lipid and a glycolipid from one or more coronaviruses selected from the group consisting of 229E, NL63, OC43, HKU1, MERS-CoV, SARS-CoV and SARS-CoV-2. In some embodiments, the coronavirus antigen is selected from the group consisting of a spike protein (S protein), a receptor-binding (RBD) protein, a S1 protein, a S2 protein, a whole protein (S1+S2), and a nucleocapsid protein (NP), from at least one coronavirus referenced above. In a specific embodiment, the coronavirus antigen is a nucleocapsid protein. In another embodiment, the coronavirus antigen is a spike protein.

In some embodiments, the first anti-coronavirus antibody is attached to a solid support to form a mobilizable conjugate. The solid support can be any material that provides a surface or a point of attachment for the first anti-coronavirus antibody and includes detectable material including, but not limited to, a colored material, a fluorescent material, and a chemiluminescent material. In one embodiment, the first anti-coronavirus antibody is attached to a solid support that includes a colloidal gold particle. In other embodiment, the anti-coronavirus antibody attached to a solid support that includes a gold nanoparticle. In other embodiments, the solid support includes a protein, such as biotin or strepavidin. In some embodiments, the first anti-coronavirus antibody of the mobilizable conjugate is capable of binding to a coronavirus antigen to form a mobilizable conjugate-antigen complex. In some embodiments, a first anti-coronavirus nucleocapsid protein IgG is attached to the surface of gold nanoparticles to form a mobilizable conjugate. In other embodiments, a first anti-coronavirus Spike protein IgG is attached to the surface of gold nanoparticles to form a mobilizable conjugate.

In some embodiments described herein, the conjugate is loaded onto to the conjugate pad and is subsequently dried. In other embodiments, a conjugation reaction is performed by mixing a lyophilized form of a capture reagent with a dry form of the solid support. In another embodiment, a lyophilized powder of a first anti-coronavirus nucleocapsid protein IgG is mixed with a dry form of gold nanoparticles to form a mobilizable conjugate. In another embodiment, a lyophilized powder of a first anti-coronavirus Spike protein IgG is mixed with a dry form of gold nanoparticles to form a mobilizable conjugate. In various embodiments described herein, a conjugation reaction to form a mobilizable conjugate comprises a conjugation reaction buffer. In a particular embodiment, the conjugation reaction buffer includes about 5 mM Potassium Phosphate and about 5 mg/mL PEG20, wherein the buffer has a pH between about 7.0-7.5. In some embodiments, the buffer comprising about 5 mM Potassium Phosphate, about 5 mg/mL PEG20, pH 7.0-7.5 is applied to the conjugate pad to rehydrate the mobilizable conjugate. In various embodiments described herein, the conjugate pads can be preblocked with a buffer solution containing a blocking buffer. In some embodiments, the purpose for preblocking the conjugate pad with a blocking buffer solution is to stabilize the conjugate when dried on the conjugate pad. In one embodiment, the blocking buffer comprises about 4 mM borate and about 1% BSA.

In various embodiments described herein, a conjugate pad is prepared by loading at least about 10 μg/mL, at least about 15 μg/mL, at least about 20 μg/mL, at least about 25 μg/mL, at least about 30 μg/mL, or at least about 35 μg/mL of the first anti-coronavirus antibody onto the conjugate pad. In one embodiment, a conjugate pad is prepared by loading at least about 15 μg/mL of the first anti-coronavirus antibody onto the conjugate pad. In certain embodiments, a conjugate pad is prepared by loading at least about 25 μg/mL of the first anti-coronavirus antibody onto the conjugate pad. In certain embodiments described herein, a conjugate pad comprises not more than about 1 μL, about 2 μL, about 5 μL, about 10 μL, or about 20 μL, of OD₂₀ mobilizable conjugate. In one embodiment, the conjugate pad comprises not more than about 10 μL, of OD₂₀ mobilizable conjugate. In another embodiment, the conjugate pad comprises not more than about 5 μL, of OD₂₀ mobilizable conjugate.

In certain embodiments of the rapid detection systems described herein, mobilizable components (e.g., a mobilizable conjugate and a mobilizable conjugate-antigen complex) migrate through the system (e.g., through the conjugate pad and through the membrane) as described herein. For example, in some embodiments, upon contacting the conjugate pad, the mobile phase (e.g., buffer comprising a sample) hydrates, suspends and/or mobilizes the mobilizable conjugate to proceed through the mobile phase by capillary action downstream through the system toward the membrane (e.g., a nitrocellulose membrane). In some embodiments, the mobilizable conjugate, upon the movement through the system by lateral flow, contacts an antigen (e.g., a coronavirus antigen) to form a complex between the mobilizable conjugate and the antigen, referred to herein as a mobilizable conjugate-antigen complex.

In various embodiments of the rapid detections systems described herein, the systems include a membrane comprising a test zone. In some embodiments, the mobilizable conjugate-antigen complex subsequently proceeds (e.g., by lateral flow) through the system from a conjugate pad into a membrane comprising a test zone. In some embodiments, a second anti-coronavirus antibody is immobilized in a region of the membrane such that the analyte binding agent is capable of bind to the mobilizable conjugate-antigen complex to form a first immobilizable complex at the test line. In preferred embodiments, the formation of the first immobilizable complex produces an observable or detectable signal at the test line indicating the presence of (and/or the amount of) analyte (e.g., a coronavirus antigen) in the sample. In some embodiments, the second anti-coronavirus antibody is immobilized to the test line through covalent or ionic means by techniques known by those skilled in the art. In certain embodiments, if an analyte of interest (e.g., a nucleocapsid protein of coronavirus) is present in the sample, the analyte of interest will bind to a cognate anti-coronavirus antibody from the mobilizable conjugate to form a mobilizable conjugate-antigen complex. In such embodiments, the mobilizable conjugate-antigen complex will be immobilized at the test line, resulting in a detectable or observable signal indicating a positive result. In other embodiments, if no analyte of interest (e.g., a nucleocapsid protein of coronavirus) is present, the mobilizable conjugate will continue to flow downstream from the test line, without immobilizing to the test line. For example, in some embodiments, the rapid detection system described herein is capable of detecting coronavirus, or a variant thereof, by the appearance of a detectable or observable line in the test zone on the membrane.

In some embodiments, the membrane comprises a test zone and a control zone, wherein the membrane is selected from the group consisting of nitrocellulose, cellulose acetate, cellulose nitrate, polyamide, polycarbonate, glass fiber, membrane, polyethersulfone, regenerated cellulose (RC), polytetra-fluorethylene, (PTFE), polyester (e.g. Polyethylene Terephthalate), polycarbonate (e.g., 4,4-hydroxy-diphenyl-2,2′-propane), Aluminum Oxide, mixed cellulose ester (e.g., mixture of cellulose acetate and cellulose nitrate), nylon (e.g., Polyamide, Hexamethylene-diamine, and Nylon 66), polypropylene, PVDF, High Density Polyethylene (HDPE)+nucleating agent “aluminum dibenzoate” (DBS). In one embodiment, the membrane is a nitrocellulose membrane.

### C. Coronavirus and Influenza Virus Rapid Detection System

The present disclosure also provides compositions for rapidly detecting and/or differentiating between a coronavirus (e.g., SARS-CoV-2) and an influenza virus in a sample (e.g., saliva sample, an oral sample, a nasal sample, a sputum sample, a blood sample). In one aspect, the present disclosure provides a rapid detection system for the detection of a coronavirus, or a variant thereof, and an influenzavirus. The system comprises a sample pad, a conjugate pad and a membrane. Although, in certain embodiments, the components of the system may be placed in various arrangements according to the assay format intended and the type of assay to be performed, generally, the components of the rapid detection system for the detection of coronavirus, or a variant thereof, and influenza virus provided herein are arranged to facilitate the unidirectional flow of the sample from the sample pad, to the conjugate pad, and to the membrane pad. In certain embodiments, the sample pad comprises a porous material. In some embodiments, the porous material comprises a matrix selected from the group consisting of a glass fiber, a nitrocellulose, a nitrocellulose blend with polyester, a nitrocellulose blend with cellulose, an untreated paper, a porous paper, and an acrylonitrile copolymer. In some embodiments, the porous material comprises a matrix comprising a glass fiber. In some embodiments described herein, the sample pad, the conjugate pad and the membrane of the rapid detection system are present in a single test strip. Common porous membranes include, but are not limited to, fiberglass, porous nitrocellulose or polyethylene. In some embodiments, a sample (e.g., a biological sample) may be obtained from a mammal (e.g., a human, a non-human primate, a dog, a cat, a horse, a pig, etc.) and selected from the group consisting of oral fluid, saliva, blood, blood plasma, blood serum, nasopharyngeal fluid, amniotic fluid, breast milk, vaginal secretions, semen, seminal fluid, urine, amniotic fluid, cerebrospinal fluid, bronchoalveolar lavage fluid, tears, mucous, tissue, tissue homogenate, cellular extract, spinal fluid, nasal fluid, and any combination of two or more of the foregoing. In yet other embodiments, the sample is a human physiological fluid, a cell culture, and an environmental sample. In some embodiments, the sample is taken from a human using a swab (e.g., a nasal swab or a throat swab). In a specific embodiment, the sample is saliva. In another embodiment, the sample is blood.

In some embodiments, a conjugate pad is composed of a hydrophobic material, such as glass fiber (e.g., Ahlstrom 8950) and comprises a first anti-coronavirus antibody attached to a first solid support to form a first mobilizable conjugate. In some embodiments, the anti-coronavirus antibody is capable of binding to a coronavirus antigen to form a mobilizable conjugate-antigen complex. In some embodiments, the coronavirus antigen includes, without limitation, a protein, a viral particle, a viral subunit, a chemical compound, a polypeptide, a carbohydrate, a nucleic acid, a lipid, and a glycolipid from one or more coronaviruses selected from the group consisting of 229E, NL63, OC43, HKU1, MERS-CoV, SARS-CoV and SARS-CoV-2. In some embodiments, the coronavirus antigen is selected from the group consisting of a spike protein (S protein), a receptor-binding (RBD) protein, a S1 protein, a S2 protein, a whole protein (S1+S2), and a nucleocapsid protein (NP), from at least one coronavirus referenced above. In a specific embodiment, the coronavirus antigen is a nucleocapsid protein. In another embodiment, the coronavirus antigen is a spike protein. The conjugate pad further comprises a first anti-influenzavirus antibody attached to a second solid support to form a second mobilizable conjugate. The anti-influenzavirus antibody is capable of binding to an influenzavirus antigen to form a second mobilizable conjugate-antigen complex. Influenza antigens may comprise, without limitation, a protein, a viral particle, a viral subunit, a polypeptide, a carbohydrate, a nucleic acid, a lipid and a glycolipid from one or more influenza viruses selected from the group consisting of influenzavirus A, influenzavirus B, influenzavirus C, and influenzavirus D.

In some embodiments, the first anti-coronavirus antibody is attached to a first solid support to form a first mobilizable conjugate. In some embodiments, the first anti-influenzavirus antibody is attached to a second solid support to form a second mobilizable conjugate. The solid support can be any material that provides a surface or a point of attachment for the first antibodies described herein and can include detectable material including, but not limited to, a colored material, a fluorescent material, and a chemiluminescent material. In one embodiment, the first anti-coronavirus antibody is attached to a solid support that includes a colloidal gold particle. In other embodiment, the first anti-coronavirus antibody attached to a solid support that includes a gold nanoparticle. In other embodiments, the solid support includes a protein, such as biotin or strepavidin. In some embodiments, a first anti-coronavirus nucleocapsid protein IgG is attached to the surface of gold nanoparticles to form a first mobilizable conjugate. In other embodiments, a first anti-coronavirus Spike protein IgG is attached to the surface of gold nanoparticles to form a first mobilizable conjugate. In one embodiment, the first anti-influenzavirus antibody is attached to a solid support that is a colloidal gold particle. In other embodiment, the first anti-influenzavirus antibody attached to a solid support that is a gold nanoparticle. In some embodiments, the first anti-coronavirus antibody and the first anti-influenzavirus antibody are both attached to the same type of solid support with the first anti-coronavirus antibody and the first anti-influenzavirus antibody attached to either a colloidal gold particle or a gold nanoparticle. In other embodiments, the first anti-coronavirus antibody and the first anti-influenzavirus antibody are both attached to a different type of solid support, with the first anti-coronavirus antibody attached to a gold nanoparticle and the first anti-influenzavirus antibody attached to a colloidal gold particle, or vice versa. In some embodiments, the first anti-coronavirus antibody of the first mobilizable conjugate is capable of binding to a coronavirus antigen to form a first mobilizable conjugate-antigen complex. In some embodiments, the first anti-influenzavirus antibody of the second mobilizable conjugate is capable of binding to an influenza antigen to form a second mobilizable conjugate-antigen complex.

In some embodiments described herein, the first and second mobilizable conjugates are loaded onto to the conjugate pad and are subsequently dried. In other embodiments, a conjugation reaction is performed by mixing a lyophilized form of a capture reagent with a dry form of the solid support. In various embodiments described herein, the conjugation reaction to form the first and second mobilizable conjugates comprise a conjugation reaction buffer. In a particular embodiment, a conjugation reaction buffer includes about 5 mM Potassium Phosphate, about 5 mg/mL PEG20, wherein the buffer has a pH between about 7.0-7.5. In some embodiments, the buffer includes about 5 mM Potassium Phosphate, about 5 mg/mL PEG20, at a pH of between about 7.0-7.5 is applied to the conjugate pad to rehydrate the first and second mobilizable conjugates. In various embodiments described herein, the conjugate pads can be preblocked with a buffer solution containing a blocking buffer. In some embodiments, the purpose for preblocking the conjugate pad with a blocking buffer solution is to stabilize the first and/or the second conjugate when dried on the conjugate pad. In one embodiment, the blocking buffer comprises about 4 mM borate and about 1% BSA.

In various embodiments described herein, a conjugate pad is prepared by loading at least about 10 μg/mL, at least about 15 μg/mL, at least about 20 μg/mL, at least about 25 μg/mL, at least about 30 μg/mL, or at least about 35 μg/mL of the first anti-coronavirus antibody onto the conjugate pad. In one embodiment, the conjugate pad is prepared by loading at least about 15 μg/mL of the first anti-coronavirus antibody onto the conjugate pad. In another embodiment, the conjugate pad is prepared by loading at least about 25 μg/mL of the first anti-coronavirus antibody onto the conjugate pad. In many embodiments described herein, the conjugate pad includes not more than about 1 μL, about 2 μL, about 5 μL, about 10 μL, or about 20 μL, of OD₂₀ mobilizable conjugate. In one embodiment, the conjugate pad comprises not more than about 10 μL, of OD₂₀ mobilizable conjugate. In another embodiment, the conjugate pad comprises not more than about 5 μL, of OD₂₀ first mobilizable conjugate.

In various embodiments described herein, the conjugate pad is prepared by further loading at least about 10 μg/mL, at least about 15 μg/mL, at least about 20 μg/mL, at least about 25 μg/mL, at least about 30 μg/mL, or at least about 35 μg/mL of the first anti-influenzavirus antibody onto the conjugate pad in addition to the corresponding equivalent amounts of the first anti-coronavirus antibody. In one embodiment, the conjugate pad is prepared by loading at least about 15 μg/mL of the first anti-coronavirus antibody and 15 μg/mL of the first anti-influenzavirus antibody onto the conjugate pad. In another embodiment, the conjugate pad is prepared by loading at least about 25 μg/mL of the first anti-coronavirus antibody and 25 μg/mL of the first anti-influenzavirus antibody onto the conjugate pad. In many embodiments described herein, the conjugate pad comprises not more than about 1 μL, about 2 μL, about 5 μL, about 10 μL, or about 20 μL, of OD₂₀ of the second mobilizable conjugate. In one embodiment, the conjugate pad comprises not more than about 10 μL, of OD₂₀ second mobilizable conjugate. In another embodiment, the conjugate pad comprises not more than about 5 μL, of OD₂₀ second mobilizable conjugate. In some embodiments, the conjugate pad comprises not more than about 20 μL, of OD₂₀ of the first and second mobilizable conjugate.

In certain embodiments of the rapid detection systems described herein, mobilizable components (e.g., a mobilizable conjugate and a mobilizable conjugate-antigen complex) migrate through the system (e.g., through the conjugate pad and membrane) as described herein. For example, in some embodiments, upon contacting the conjugate pad, the mobile phase (e.g., a buffer or a sample) hydrates, suspends and/or mobilizes the mobilizable conjugate(s) to proceed through the mobile phase by capillary action downstream through the system toward the membrane (e.g., a nitrocellulose membrane).

In various embodiments of the rapid detection systems described herein, the systems include a membrane comprising a test zone and a control zone. In some embodiments, the first mobilizable conjugate-antigen complex and second mobilizable conjugate-antigen complex, subsequently proceed (e.g., by lateral flow) through the system from a conjugate pad into a membrane comprising a first test zone, a second test zone, and at least one control zone. In some embodiments, the first test zone comprises a second anti-coronavirus antibody immobilized to the membrane. The second anti-coronavirus antibody is capable of binding to the first mobilizable conjugate-antigen complex to form a first immobilizable complex. In preferred embodiments, the formation of the first immobilizable complex produces an observable or detectable signal at the first test line indicating the presence of (and, in some embodiments, the amount of) analyte (e.g., a coronavirus antigen) in the sample. If no coronavirus antigen is present in the sample, the first mobilizable conjugate will continue to travel downstream to the control line without binding at the first test line. In some embodiments, the second test zone comprises a second anti-influenzavirus antibody immobilized to the membrane. The second anti-influenzavirus antibody is capable of binding to the second mobilizable conjugate-antigen complex to form a second immobilizable complex. In preferred embodiments, the formation of the second immobilizable complex produces an observable or detectable signal at the second test line indicating the presence of (and, in some embodiments, the amount of) analyte (e.g., an influenza virus antigen) in the sample. If no influenza antigen is present in the sample, the second mobilizable conjugate will continue to travel downstream to the control line without binding at the second test line. For example, the rapid detection system described herein can simultaneously detect a coronavirus, or a variant thereof, and an influenzavirus by the appearance of a detectable or observable line in one or in both the first and the second test zones, respectively, on the membrane simultaneously.

In some embodiments, the membrane comprises at least one control zone. The control zones typically comprise an immunoglobulin immobilized to the membrane. In some embodiments, the membrane comprises only one control zone. In some embodiments, the membrane comprises a first control zone and a second control zone. In one embodiment, the immunoglobulin is an anti-human IgG molecule (or variant thereof) or an anti-mouse IgG molecule (or variant thereof) that is capable of binding to the first mobilizable conjugate to form a third immobilizable complex. The formation of the third immobilizable complex at the control line, typically, produces an observable or detectable signal, indicating the liquid sample has permeated or flowed through the matrix as designed. In another embodiment, the immunoglobulin is an anti-human IgG molecule (or variant thereof) or anti-mouse IgG molecule (or variant thereof) or that is capable of binding to the second mobilizable conjugate to form a fourth immobilizable complex. The formation of the fourth immobilizable complex at the control line, typically, produces an observable or detectable signal, indicating the liquid sample has permeated or flowed through the matrix as designed. In some embodiments, the immunoglobulin striped at the first control zone and the second control zone is an anti-human or anti-mouse IgG molecule that is capable of binding to the first and second mobilizable conjugate, respectively, to form the third immobilizable complex at the first control zone and to form the fourth third immobilizable complex at the first control zone. The formation of the third immobilizable complex at the first control zone, and the formation of the fourth immobilizable complex at the second control zone, respectively, produces detectable signals, indicating that the liquid sample has permeated or flowed through the matrix as designed.

In some embodiments, the membrane comprises a first test zone, a second test zone, and at least one control zone, wherein the membrane is selected from the group consisting of nitrocellulose, cellulose acetate, cellulose nitrate, polyamide, polycarbonate, glass fiber, membrane, polyethersulfone, regenerated cellulose (RC), polytetra-fluorethylene, (PTFE), polyester (e.g. Polyethylene Terephthalate), polycarbonate (e.g., 4,4-hydroxy-diphenyl-2,2′-propane), Aluminum Oxide, mixed cellulose ester (e.g., mixture of cellulose acetate and cellulose nitrate), nylon (e.g., Polyamide, Hexamethylene-diamine, and Nylon 66), polypropylene, PVDF, High Density Polyethylene (HDPE)+nucleating agent “aluminum dibenzoate” (DBS). In one embodiment, the membrane is a nitrocellulose membrane.

In general, the rapid detection systems described herein can comprise a test strip such as the exemplary test strip with a schematic of the sectional view shown in FIGS. 1 and 2 that includes a matrix defining an axial flow path. Typically, the matrix includes a sample pad, conjugate pad, and a membrane (e.g., a nitrocellulose membrane) with one or more test zones and one or more control zones, a wick pad and a backing card. The systems described herein can include other devices (such as but not limited to a test strip), a housing including a port configured to receive all or a portion of the test device, a reader including a light source and a light detector, a data analyzer, and combinations thereof. The rapid detection systems may be encased or housed in a casing made of a wide variety of materials, including plastic, metal, or composite materials. The housing forms a protective enclosure for components of the rapid detection systems. In some embodiments, the casing is a portable device that allows for the ability to perform a lateral flow assay in a variety of environments, including on the bench, in the field, in the home, or in a facility for domestic, commercial, or environmental applications.

In some of the contemplated embodiments, multiple types of reagents are incorporated into the rapid detection systems described herein above, such that they may flow by capillary action together with a sample. These multiple types of reagents can be analyte specific (e.g., antibodies specifically binding to the antigens in the sample) or control reagents and may have different detectable characteristics (e.g., different colors) such that one labeled reagent can be differentiated from another labeled reagent if utilized in the same detection systems, or in any embodiment described herein, having different capture moieties. As the reagents are frequently bound to a specific analyte of interest, differential detection of the reagents having different specificities (including analyte specific and control reagents) may be a desirable attribute.

### II. Methods for Rapid Detection of Antigens

The present disclosure, also provides methods that utilize the rapid detection systems described herein for the for the qualitative and/or quantitative detection of one or more viral antigens in a sample. Exemplary viral antigens include antigens from a double-stranded RNA virus, a positive sense RNA virus, a negative sense RNA virus, a retrovirus, such as a Coronaviridae virus, a Picornaviridae virus, a Caliciviridae virus, a Flaviviridae virus, a Togaviridae virus, a Bornaviridae, a Filoviridae, a Paramyxoviridae, a Pneumoviridae, a Rhabdoviridae, an Arenaviridae, a Bunyaviridae, an Orthomyxoviridae, or a Deltavirus. The methods provided herein can be used to detect a wide variety of viral antigens including but not limited to a SARS-CoV-2 antigen, a coronavirus antigen, an influenza antigen, and any combinations thereof, in a biological sample.

### A. Method for Rapid Detection of SARS-CoV-2

The present disclosure provides a method for rapid detection of SARS-CoV-2, or a variant thereof, in a biological sample (e.g., an oral sample, a mucus sample, a nasal sample, a sputum sample). In one aspect, the method comprises the steps of dispersing a biological sample suspected of having a SARS-CoV-2 antigen in a sample buffer, contacting the biological sample dispersed in the sample buffer with a sample pad comprising a porous material through which the biological sample can flow by capillary action, and contacting the liquid front of the sample flowing from the sample pad with a conjugate pad. In one embodiment, the conjugate pad comprises a first anti-SARS-CoV-2 antibody attached to a solid support to form a mobilizable conjugate. In some embodiments, the solid support is a gold nanoparticle. Methods of immobilizing an antibody on a membrane (e.g., nitrocellulose membrane) are well known to a person of skill in the art. In the various embodiments of the method, the first anti-SARS-CoV-2 antibody of the mobilizable conjugate is capable of binding to the SARS-CoV-2 antigen to form a mobilizable conjugate-antigen complex, and the contacting is performed under conditions that permit the capillarity of the mobilizable conjugate. The method further comprises contacting the mobilizable conjugate-antigen complex with a membrane comprising a test zone. In some embodiments, the test zone comprises a second anti-SARS-CoV-2 antibody immobilized to the membrane. Methods of immobilizing an antibody on a membrane (e.g., nitrocellulose membrane) are well known to a person of skill in the art. In some embodiments of the method, the second anti-SARS-CoV-2 antibody is capable of binding to the mobilizable conjugate-antigen complex to form a first immobilizable complex. In some embodiments, the first immobilizable complex produces a detectable signal at the test zone indicating the presence of the SARS-CoV-2 antigen in the biological sample. In other embodiments of the method, the mobilizable conjugate and/or the mobilizable conjugate-antigen complex comprises a detectable agent that produces a detectable signal upon binding to the second anti-SARS-CoV-2 antibody at the test zone, indicating the presence of the SARS-CoV-2 antigen in the biological sample. In yet another embodiment of the method, the second anti-SARS-CoV-2 antibody is conjugated to a detectable agent capable of producing a detectable signal at the test zone.

In some embodiments of the method described herein, the membrane further comprises a control zone that is located in a spatially distinct region of the membrane from a test zone. In some embodiments, the control zone is located downstream from the test zone. In such embodiments, the control zone comprises an immunoglobulin immobilized to the membrane wherein the immunoglobulin is capable of binding to the first anti-SARS-CoV-2 antibody to form a second immobilizable complex. The formation of the second immobilizable complex produces a detectable signal at the control zone indicating capillarity of the biological sample.

In some embodiments, the detectable signals disclosed in the methods described above may include a visible signal that can be detected without the use of any additional instrumentation (or instrumentation transduction). In some embodiments, the visible signal is a line (e.g., a colored line) formed at the test and/or the control zones as described herein. In some embodiments, the antibodies are preferably conjugated with one or more detectable agents, such as biotin, fluorescein, ruthenium, europium, gold particles, alkaline phosphatase, galactosidase, or horseradish peroxidase. However, other suitable agents or detection systems can also be employed. In other embodiments, the detectable signals described herein may also be compatible with luminescence, phosphorescence, and light scattering based signal transduction. In exemplary embodiments, excitable tags conjugated to one or more of the mobilizable conjugate, the mobilizable conjugate-antigen complex and the second anti-SARS-CoV-2 antibody, may be used as detection reagents in the methods described herein. Exemplary tags include, but are not limited to, nanoparticles such as gold nanoparticles, fluorescent organic dyes such as fluorescein, rhodamine, and commercial derivatives such as Alexa dyes (Life Technologies) and DyLight products; fluorescent proteins such as R-phycoerythrin and commercial analogs such as SureLight P3; luminescent lanthanide chelates; luminescent semiconductor nanoparticles (e.g., quantum dots); phosphorescent materials, and microparticles that incorporate these excitable tags. For the purpose of this disclosure, the term “fluorophore” is used generically to describe all of the excitable tags listed above.

In some embodiments of the methods described herein, the mobilizable conjugate is capable of binding to a SARS-CoV-2 antigen selected from the group consisting of a spike protein (S), a receptor-binding (RBD) protein, a S1 protein, a S2 protein, a whole protein (S1+S2), and a nucleocapsid protein (NP). In a specific embodiment, the SARS-CoV-2 antigen is a nucleocapsid protein. In another embodiment, the SARS-CoV-2 antigen is a spike protein. In some embodiments of the method, a sample may be obtained from a mammal (e.g., a human, a non-human primate, a dog, a cat, a horse, a pig, etc.) and selected from the group consisting of oral fluid, saliva, blood, blood plasma, blood serum, nasopharyngeal fluid, amniotic fluid, breast milk, vaginal secretions, semen, seminal fluid, urine, amniotic fluid, cerebrospinal fluid, bronchoalveolar lavage fluid, tears, mucous, tissue, tissue homogenate, cellular extract, spinal fluid, and any combination of two or more of the foregoing. In yet other embodiments, the sample is a human physiological fluid, a cell culture, and an environmental sample(s). In some embodiments of the method, the sample is taken from a human using a swab (e.g., a nasal swab or throat swab). In a specific embodiment of the method, the sample is saliva. In another embodiment of the method, the sample is blood.

In some embodiments of the method described herein, the flow of the sample through the sample pad is frequently facilitated by the addition of a buffer. In the various embodiments of the method described herein, the sample is dissolved, dispersed, suspended and/or reconstituted in one or more appropriate buffers. In some embodiments, the sample is dispersed in a sample buffer prior to loading onto a sample pad. In other embodiments, the sample is loaded directly onto a sample pad that has been pre-treated with the sample buffer. In certain embodiments of the method described herein, the sample buffer comprises a phosphate buffered saline solution (PBS). In specific embodiment of the method, the Ahlstrom grade 8950 glass fiber sample pads are pretreated with 10×PBS. In some embodiments of the method, the 10×PBS are prepared from packets acquired through Sigma Aldrich or Thermo Fisher and applied to the Ahlstrom grade 8950 glass fiber sample pads. In particular embodiment of the method, the Ahlstrom 8950 sample pads pretreated with 10×PBS exhibited less non-specific binding compared to than those prepared with other sample pads. In some embodiments of the methods described herein, the pretreated sample pads appeared to improve the test performance, allowing signal modulation between negative samples and samples spiked to 0.0001 μg/mL nucleocapsid protein (FIG. 5). In some other embodiments, the method further comprises a blocking step. The blocking step comprises contacting the conjugation pad with a blocking buffer for at least 30 minutes prior to contacting the biological sample dispersed in the sample buffer with a sample pad. In a particular embodiment, the blocking buffer includes about 4 mM borate and about 1% BSA. A sensitivity close to 0.0001 μg/mL was achieved using the normal pooled saliva (NPS) sample using the conditions outlined in Table 1.

In various embodiments of the method described herein, the first anti-SARS-CoV-2 antibody is Antibody B (Clone B3451M). In some embodiments, the second anti-SARS-CoV-2 antibody is Antibody A (Clone B3449M). In one embodiment, the first anti-SARS-CoV-2 antibody is Antibody B (Clone B3451M) and the second anti-SARS-CoV-2 antibody is Antibody A (Clone B3449M). In another embodiment, the first anti-SARS-CoV-2 antibody is an anti-SARS-CoV-2 (ARISTA Biologicals.) In some embodiments of the method, the conjugate pad is prepared by loading at least about 10 μg/mL, at least about 15 μg/mL, at least about 20 μg/mL, at least about 25 μg/mL, at least about 30 μg/mL, or at least about 35 μg/mL of the first anti-SARS-CoV-2 antibody onto the conjugate pad. In one embodiment, the conjugate pad is prepared by loading at least about 15 μg/mL of the first anti-SARS-CoV-2 antibody onto the conjugate pad. In another embodiment, the conjugate pad is prepared by loading at least about 25 μg/mL of the first anti-SARS-CoV-2 antibody onto the conjugate pad. In many embodiments described herein, the conjugate pad comprises not more than about 1 μL, about 2 μL, about 5 μL, about 10 μL, or about 20 μL, of OD₂₀ mobilizable conjugate. In one embodiment, the conjugate pad comprises not more than about 10 μL, of OD₂₀ mobilizable conjugate. In another embodiment, the conjugate pad comprises not more than about 5 μL, of OD₂₀ mobilizable conjugate. In some embodiments of the method, the second anti-SARS-CoV-2 antibody is immobilized on the membrane at a concentration of between about 10 μg/mL to 30 μg/mL. In one embodiment of the method, the second anti-SARS-CoV-2 antibody is immobilized on the membrane at a concentration of about 15 μg/mL. In one embodiment of the method, the first anti-SARS-CoV-2 antibody is immobilized on the first solid support at a concentration of about 15 μg/mL and the second anti-SARS-CoV-2 antibody is immobilized on the membrane at a concentration of about 15 μg/mL.

In certain embodiments of the method, the membrane comprises a test zone and a control zone, wherein the membrane is selected from the group consisting of nitrocellulose, cellulose acetate, cellulose nitrate, polyamide, polycarbonate, glass fiber, membrane, polyethersulfone, regenerated cellulose (RC), polytetra-fluorethylene, (PTFE), polyester (e.g. Polyethylene Terephthalate), polycarbonate (e.g., 4,4-hydroxy-diphenyl-2,2′-propane), Aluminum Oxide, mixed cellulose ester (e.g., mixture of cellulose acetate and cellulose nitrate), nylon (e.g., Polyamide, Hexamethylene-diamine, and Nylon 66), polypropylene, PVDF, High Density Polyethylene (HDPE)+nucleating agent “aluminum dibenzoate” (DBS). In one embodiment, the membrane is a nitrocellulose membrane. In a specific embodiment, the nitrocellulose membrane is a MDI10μ-CNPF membrane. In another embodiment, the nitrocellulose membrane is a MDI70-CNPH membrane. In various embodiments described herein, the test lines are striped at least about 0.04 μL/mm, at least about 0.08 μL/mm, at least about 0.12 μL/mm with an antibody concentration of about 0.1 mg/mL, about 0.5 mg/mL, about 1.0 mg/mL or about 2.0 mg/mL.

### B. Method for Rapid Detection of Coronavirus

The present disclosure provides a method for the rapid detection of a coronavirus in a biological sample (e.g., an oral sample, a mucus sample, a nasal sample, a sputum sample). In one aspect, the method comprises the steps of dispersing a biological sample suspected of having a coronavirus antigen in a sample buffer, contacting the biological sample dispersed in the sample buffer with a sample pad comprising a porous material through which the biological sample can flow by capillary action, and contacting the liquid front of the sample flowing from the sample pad with a conjugate pad. In one embodiment, the conjugate pad comprises a first anti-coronavirus antibody attached to a solid support to form a mobilizable conjugate. In some embodiments, the solid support is a gold nanoparticle. In the various embodiments of the method, the first anti-coronavirus antibody of the mobilizable conjugate is capable of binding to the coronavirus antigen to form a mobilizable conjugate-antigen complex, and the contacting is performed under conditions that permit the capillarity of the mobilizable conjugate. The method further comprises contacting the mobilizable conjugate-antigen complex with a membrane comprising a test zone. In some embodiments, the test zone comprises a second anti-coronavirus antibody immobilized to the membrane. Methods of immobilizing an antibody on a membrane (e.g., nitrocellulose membrane) are well known to a person of skill in the art. In some embodiments of the method, the second anti-coronavirus antibody is capable of binding to the mobilizable conjugate-antigen complex to form a first immobilizable complex. In some embodiments, the first immobilizable complex produces a detectable signal at the test zone indicating the presence of the coronavirus antigen in the biological sample. In other embodiments of the method, the mobilizable conjugate and/or the mobilizable conjugate-antigen complex comprises a detectable agent that produces a detectable signal upon binding to the second anti-coronavirus antibody at the test zone, indicating the presence of the coronavirus antigen in the biological sample. In yet another embodiment of the method, the second anti-coronavirus antibody is conjugated to a detectable agent capable of producing a detectable signal at the test zone.

In some embodiments of the method described herein, the membrane further comprises a control zone that is located in a spatially distinct region of the membrane from a test zone. In some embodiments, the control zone is located downstream from the test zone. In such embodiments, the control zone comprises an immunoglobulin immobilized to the membrane wherein the immunoglobulin is capable of binding to the first anti-coronavirus antibody to form a second immobilizable complex. The formation of the second immobilizable complex produces a detectable signal at the control zone indicating capillarity of biological sample.

In some embodiments, the detectable signals disclosed in the various embodiments of the method described above may include a visible signal that can be detected without the use of any additional instrumentation (or instrumentation transduction). In some embodiments, the visible signal is a line (e.g., a colored line) formed at the test and/or control zones described in the methods provided herein. In some embodiments, the antibodies are preferably conjugated with one or more detectable agents, such as biotin, fluorescein, ruthenium, europium, gold particles, alkaline phosphatase, galactosidase, or horseradish peroxidase. However, other suitable agents or detection systems can also be employed. In other embodiments, the detectable signals described herein may also be compatible with luminescence, phosphorescence, and light scattering based signal transduction. In exemplary embodiments, excitable tags conjugated to one or more of the mobilizable conjugate, the mobilizable conjugate-antigen complex and the second anti-coronavirus antibody, may be used as detection reagents in the methods described herein. Exemplary tags include, but are not limited to, nanoparticles such as gold nanoparticles, fluorescent organic dyes such as fluorescein, rhodamine, and commercial derivatives such as Alexa dyes (Life Technologies) and DyLight products; fluorescent proteins such as R-phycoerythrin and commercial analogs such as SureLight P3; luminescent lanthanide chelates; luminescent semiconductor nanoparticles (e.g., quantum dots); phosphorescent materials, and microparticles that incorporate these excitable tags. For the purpose of this disclosure, the term “fluorophore” is used generically to describe all of the excitable tags listed above.

In some embodiments of the methods described herein, the mobilizable conjugate is capable of binding to a coronavirus antigen selected from the group consisting of a spike protein (S), a receptor-binding (RBD) protein, a S1 protein, a S2 protein, a whole protein (S1+S2), and a nucleocapsid protein (NP) of at least one coronavirus selected from the group consisting of 229E, NL63, OC43, HKU1, MERS-CoV, SARS-CoV and SARS-CoV-2. In a specific embodiment, the coronavirus antigen is a nucleocapsid protein. In another embodiment, the coronavirus antigen is a spike protein. In some embodiments of the method, a sample may be obtained from a mammal (e.g., a human, a non-human primate, a dog, a cat, a horse, a pig, etc.) and selected from the group consisting of oral fluid, saliva, blood, blood plasma, blood serum, nasopharyngeal fluid, amniotic fluid, breast milk, vaginal secretions, semen, seminal fluid, urine, amniotic fluid, cerebrospinal fluid, bronchoalveolar lavage fluid, tears, mucous, tissue, tissue homogenate, cellular extract, spinal fluid, and any combination of two or more of the foregoing. In yet other embodiments, the sample is a human physiological fluid, a cell culture, and an environmental sample. In some embodiments of the method, the sample is taken from a human using a swab (e.g., a nasal swab or throat swab). In a specific embodiment of the method, the sample is saliva. In another embodiment of the method, the sample is blood.

In some embodiments of the method described herein, the flow of the sample through the sample pad is frequently facilitated by the addition of a buffer. In the various embodiments of the method described herein, the sample is dissolved, dispersed, suspended and/or reconstituted in one or more appropriate buffers. In some embodiments, the sample is dispersed in a sample buffer prior to loading onto a sample pad. In other embodiments, the sample is loaded directly onto a sample pad that has been pre-treated with the sample buffer. In certain embodiments of the method described herein, the sample buffer comprises a phosphate buffered saline solution (PBS). In specific embodiment of the method, the Ahlstrom grade 8950 glass fiber sample pads were pretreated with 10×PBS. In some embodiments of the method, the 10×PBS was prepared from packets acquired through Sigma Aldrich or Thermo Fisher and applied to the Ahlstrom grade 8950 glass fiber sample pads. In particular embodiment of the method, the Ahlstrom 8950 sample pads pretreated with 10×PBS exhibited less non-specific binding compared to than those prepared with other sample pads. In some embodiments of the methods described herein, the pretreated sample pads appeared to improve the test performance, allowing signal modulation between negative samples and samples spiked to 0.0001 μg/mL nucleocapsid protein (FIG. 5). In some other embodiments, the method further comprises a blocking step. The blocking step comprises contacting the conjugation pad with a blocking buffer for at least 30 minutes prior to contacting the biological sample dispersed in the sample buffer with a sample pad. In a particular embodiment, the blocking buffer comprises about 4 mM borate and about 1% BSA. A sensitivity close to 0.0001 μg/mL was achieved using the normal pooled saliva (NPS) sample using the conditions outlined in Table 1.

In various embodiments of the method described herein, the conjugate pad is prepared by loading at least about 10 μg/mL, at least about 15 μg/mL, at least about 20 μg/mL, at least about 25 μg/mL, at least about 30 μg/mL, or at least about 35 μg/mL of the first anti-coronavirus antibody onto the conjugate pad. In one embodiment, the conjugate pad is prepared by loading at least about 15 μg/mL of the first anti-coronavirus antibody onto the conjugate pad. In another embodiment, the conjugate pad is prepared by loading at least about 25 μg/mL of the first anti-coronavirus antibody onto the conjugate pad. In many embodiments described herein, the conjugate pad comprises not more than about 1 μL, about 2 μL, about 5 μL, about 10 μL, or about 20 μL, of OD₂₀ mobilizable conjugate. In one embodiment, the conjugate pad comprises not more than about 10 μL, of OD₂₀ mobilizable conjugate. In another embodiment, the conjugate pad comprises not more than about 5 μL, of OD₂₀ mobilizable conjugate. In some embodiments of the method, the second anti-coronavirus antibody is immobilized on the membrane at a concentration of between about 10 μg/mL to 30 μg/mL. In one embodiment of the method, the second anti-coronavirus antibody is immobilized on the membrane at a concentration of about 15 μg/mL. In one embodiment of the method, the first anti-coronavirus antibody is immobilized on the first solid support at a concentration of about 15 μg/mL and the second anti-coronavirus antibody is immobilized on the membrane at a concentration of about 15 μg/mL.

In certain embodiments of the method, the membrane comprises a test zone and a control zone, wherein the membrane is selected from the group consisting of nitrocellulose, cellulose acetate, cellulose nitrate, polyamide, polycarbonate, glass fiber, membrane, polyethersulfone, regenerated cellulose (RC), polytetra-fluorethylene, (PTFE), polyester (e.g. Polyethylene Terephthalate), polycarbonate (e.g., 4,4-hydroxy-diphenyl-2,2′-propane), Aluminum Oxide, mixed cellulose ester (e.g., mixture of cellulose acetate and cellulose nitrate), nylon (e.g., Polyamide, Hexamethylene-diamine, and Nylon 66), polypropylene, PVDF, High Density Polyethylene (HDPE)+nucleating agent “aluminum dibenzoate” (DBS). In one embodiment, the membrane is a nitrocellulose membrane. In a specific embodiment, the nitrocellulose membrane is a MDI10μ-CNPF membrane. In another embodiment, the nitrocellulose membrane is a MDI70-CNPH membrane. In various embodiments described herein, the test lines are striped at least about 0.04 μL/mm, at least about 0.08 μL/mm, at least about 0.12 μL/mm with an antibody concentration of about 0.1 mg/mL, about 0.5 mg/mL, about 1.0 mg/mL or about 2.0 mg/mL.

### C. Method for Rapid Detection of Coronavirus and an Influenzavirus

The present disclosure also provides a method for rapid detection of a coronavirus (e.g., SARS-CoV-2), or a variant thereof, and an influenzavirus in a biological sample (e.g., an oral sample, a mucus sample, a nasal sample, a sputum sample). In one aspect, the method comprises the steps of a biological sample suspected of having either a coronavirus antigen or an influenzavirus antigen in a sample buffer, contacting the biological sample dispersed in the sample buffer with a sample pad comprising a porous material through which the biological sample can flow by capillary action, and contacting the liquid front of the sample flowing from the sample pad with a conjugate pad. In one embodiment of the method, the biological sample has both a coronavirus antigen and an influenzavirus antigen. In one embodiment, the conjugate pad comprises a first anti-coronavirus antibody attached to a first solid support to form a first mobilizable conjugate. The first mobilizable conjugate is capable of binding to the coronavirus antigen to form a first mobilizable conjugate-antigen complex. The conjugate pad of the method further comprises a second solid support to which a first anti-influenzavirus antibody is attached, forming a second mobilizable conjugate. The second mobilizable conjugate is capable of binding to the influenzavirus antigen to form a second mobilizable antibody-antigen complex. In some embodiments, the first and second solid supports are identical comprising a gold nanoparticle. In other embodiments, the first and second solid supports are different. In a particular embodiment where the first and second solid supports are different, at least one of the solid supports comprise a gold nanoparticle. In the various embodiments of the method, the first anti-coronavirus antibody of the first mobilizable conjugate is capable of binding to a coronavirus antigen to form a first mobilizable conjugate-antigen complex, and the contacting is performed under conditions that permit the capillarity of the mobilizable conjugate. In various other embodiments of the method, the first anti-influenzavirus antibody of the mobilizable conjugate is capable of binding to the coronavirus antigen to form a mobilizable conjugate-antigen complex, and the contacting is performed under conditions that permit the capillarity of the mobilizable conjugate.

The method further comprises contacting the first and second mobilizable conjugate-antigen complexes with a membrane. In various embodiments, the membrane comprises a first and second test zones, which are capable of capturing the first and second mobilizable conjugate-antigen complexes, respectively. In one embodiment, the first test zone comprises a second anti-coronavirus antibody immobilized to the membrane. Methods of immobilizing an antibody on a membrane (e.g., nitrocellulose membrane) are well known to a person of skill in the art. In some embodiments of the method, the second anti-coronavirus antibody is capable of binding to the first mobilizable conjugate-antigen complex to form a first immobilizable complex. In some embodiments, the first immobilizable complex produces a detectable signal at the test zone indicating the presence of the coronavirus antigen in the biological sample. In various embodiments, the second test zone comprises a second anti-influenzavirus antibody immobilized to the membrane. In some embodiments of the method, the second anti-influenzavirus antibody is capable of binding to the second mobilizable conjugate-antigen complex to form a second immobilizable complex. In the embodiments of the method with the second test zone, the second immobilizable complex produces a detectable signal at the second test zone indicating the presence of the influenzavirus antigen in the biological sample. In some embodiments of the method, the first immobilizable complex produces a detectable signal at the first test zone and the second immobilizable complex produces a detectable signal at the second test zone, indicating the presence of both the coronavirus and the influenzavirus antigen in the biological sample.

In some embodiments of the method, the first and second mobilizable conjugate and/or the first and second mobilizable conjugate-antigen complexes comprise a detectable agent that produces a detectable signal upon binding to the second anti-coronavirus antibody at the first test zone and the second anti-influenzavirus antibody at the second test zone, respectively. In specific embodiments the detectable agents attached to the first and second mobilizable conjugate and/or the first and second mobilizable conjugate-antigen complex, are identical, comprising nanogold particles. In another embodiment the detectable agents attached to the first and second mobilizable conjugate and the first and second mobilizable conjugate-antigen complexes, are different. In yet another embodiment of the method, the second anti-coronavirus antibody and the second anti-influenzavirus antibody, are conjugated to a detectable agent capable of producing a detectable signal at the first and second test zones, respectively.

In some embodiments of the method described herein, the membrane further comprises at least one control zone that is located in a spatially distinct region of the membrane from a test zone. In some embodiments, the control zone is located downstream from the test zone. In such embodiments, the at least one control zone comprises at least one immunoglobulin immobilized to the membrane, wherein the immunoglobulin is capable of binding to the first mobilizable conjugate and the second mobilizable conjugate. In one embodiment, the membrane comprises one control zone downstream of the test zone such that an anti-human immunoglobulin immobilized to the membrane. The anti-human immunoglobulin is capable of binding to the first anti-coronavirus antibody in the first mobilizable conjugate and the first anti-influenzavirus antibody in the second mobilizable conjugate, indicating capillarity of the biological sample.

In some embodiments of the method described herein, the membrane further comprises a first control zone and a second control zone. The first control zone and the second control zone are downstream from the first test zone and the second test zone. The first control zone comprises a first immunoglobulin immobilized to the membrane. The first immunoglobulin is capable of binding to the first mobilizable conjugate to form the third immobilizable complex. The third immobilizable complex produces a third detectable signal at the first control zone indicating capillarity of the biological sample, serving as an internal control for the sample comprising the coronavirus antigen. The second control zone comprises a second immunoglobulin immobilized to the membrane. The second immunoglobulin is capable of binding to the second mobilizable conjugate to form the fourth immobilizable complex. The fourth immobilizable complex produces a fourth detectable signal at the second control zone indicating capillarity of the biological sample, serving as an internal control for the sample comprising the influenzavirus antigen.

In some embodiments, the detectable signals disclosed in the various embodiments of the method described above may include a visible signal that can be detected without the use of any additional instrumentation (or instrumentation transduction). In some embodiments, the visible signal is a line (e.g., a colored line) formed at the test zone and/or at the control zones described herein. In some embodiments, the antibodies are preferably conjugated with one or more detectable agents, such as biotin, fluorescein, ruthenium, europium, gold particles, alkaline phosphatase, galactosidase, or horseradish peroxidase. However, other suitable agents or detection systems can also be employed. In other embodiments, the detectable signals described herein may also be compatible with luminescence, phosphorescence, and light scattering based signal transduction. In exemplary embodiments, excitable tags conjugated to one or more of the mobilizable conjugate, the mobilizable conjugate-antigen complex and the second anti-coronavirus antibody, may be used as detection reagents in the methods described herein. Exemplary tags include, but are not limited to, nanoparticles such as gold nanoparticles, fluorescent organic dyes such as fluorescein, rhodamine, and commercial derivatives such as Alexa dyes (Life Technologies) and DyLight products; fluorescent proteins such as R-phycoerythrin and commercial analogs such as SureLight P3; luminescent lanthanide chelates; luminescent semiconductor nanoparticles (e.g., quantum dots); phosphorescent materials, and microparticles that incorporate these excitable tags. For the purpose of this disclosure, the term “fluorophore” is used generically to describe all of the excitable tags listed above.

In some embodiments of the methods described herein, the first anti-coronavirus antibody is capable of binding to at least one coronavirus selected from the group consisting of 229E, NL63, OC43, HKU1, MERS-CoV, SARS-CoV and SARS-CoV-2. In some other embodiments of the method, the second anti-coronavirus antibody is capable of binding to at least one coronavirus selected from the group consisting of 229E, NL63, OC43, HKU1, MERS-CoV, SARS-CoV and SARS-CoV-2. In one embodiment of the method, the first and the second anti-coronavirus antibodies are capable of binding to at least one coronavirus selected from the group consisting of 229E, NL63, OC43, HKU1, MERS-CoV, SARS-CoV and SARS-CoV-2. In a specific embodiment, the first and the second anti-coronavirus antibodies are capable of binding to the same type of coronavirus selected from the group consisting of 229E, NL63, OC43, HKU1, MERS-CoV, SARS-CoV and SARS-CoV-2. In some embodiments, the first mobilizable conjugate is capable of binding to the coronavirus antigen selected from the group consisting of a spike protein (S), a receptor-binding (RBD) protein, a S1 protein, a S2 protein, a whole protein (S1+S2), and a nucleocapsid protein (NP) of at least one coronavirus selected from the group consisting of 229E, NL63, OC43, HKU1, MERS-CoV, SARS-CoV and SARS-CoV-2. In a specific embodiment, the coronavirus antigen is a nucleocapsid protein. In another embodiment, the coronavirus antigen is a spike protein.

In some embodiments of the method, the first anti-influenzavirus antibody is capable of binding to at least one influenzavirus antigen selected from the group consisting of an influenzavirus A antigen, an influenzavirus B antigen, an influenzavirus C antigen, and an influenzavirus D antigen. In some other embodiments of the method, the second anti-influenzavirus antibody is capable of binding to at least one influenzavirus antigen selected from the group consisting of an influenzavirus A antigen, an influenzavirus B antigen, an influenzavirus C antigen, and an influenzavirus D antigen. In one embodiment of the method, the first and the second anti-influenzavirus antibodies are capable of binding to at least one influenzavirus antigen selected from the group consisting of an influenzavirus A antigen, an influenzavirus B antigen, an influenzavirus C antigen, and an influenzavirus D antigen. In a specific embodiment, the first and the second anti-influenzavirus antibodies are capable of binding to the same type of influenzavirus antigen selected from the group consisting of an influenzavirus A antigen, an influenzavirus B antigen, an influenzavirus C antigen, and an influenzavirus D antigen.

In some embodiments of the method, a sample suspected of having either a coronavirus antigen or an influenzavirus antigen, may be obtained from a mammal (e.g., a human, a non-human primate, a dog, a cat, a horse, a pig, etc.) and selected from the group consisting of oral fluid, saliva, blood, blood plasma, blood serum, nasopharyngeal fluid, amniotic fluid, breast milk, vaginal secretions, semen, seminal fluid, urine, amniotic fluid, cerebrospinal fluid, bronchoalveolar lavage fluid, tears, mucous, tissue, tissue homogenate, cellular extract, spinal fluid, and any combination of two or more of the foregoing. In yet other embodiments, the sample is a human physiological fluid, a cell culture, or an environmental sample(s). In some embodiments of the method, the sample is taken from a human using a swab (e.g., a nasal swab or throat swab). In a specific embodiment of the method, the sample is saliva. In another embodiment of the method, the sample is blood.

In some embodiments of the method described herein, the flow of the sample through the sample pad is frequently facilitated by the addition of a buffer. In the various embodiments of the method described herein, the sample is dissolved, dispersed, suspended and/or reconstituted in appropriate buffers. In some embodiments, the sample is dispersed in a sample buffer prior to loading onto the sample pad. In other embodiments, the sample is loaded directly onto a sample pad that has been pre-treated with the sample buffer. In certain embodiments of the method described herein, the sample buffer comprises a phosphate buffered saline solution (PBS). In specific embodiment of the method, the Ahlstrom grade 8950 glass fiber sample pads were pretreated with 10×PBS. In some embodiments of the method, the 10×PBS was prepared from packets acquired through Sigma Aldrich or Thermo Fisher and applied to the Ahlstrom grade 8950 glass fiber sample pads. In particular embodiment of the method, the Ahlstrom 8950 sample pads pretreated with 10×PBS exhibited less non-specific binding compared to than those prepared with other sample pads. In some embodiments of the methods described herein, the pretreated sample pads appeared to improve the test performance, allowing signal modulation between negative samples and samples spiked to 0.0001 μg/mL nucleocapsid protein (FIG. 5). In some other embodiments, the method further comprises a blocking step. The blocking step comprises contacting the conjugation pad with a blocking buffer for at least 30 minutes prior to contacting the biological sample dispersed in the sample buffer with a sample pad. In a particular embodiment, the blocking buffer comprises about 4 mM borate and about 1% BSA. A sensitivity close to 0.0001 μg/mL was achieved using the normal pooled saliva (NPS) sample using the conditions outlined in Table 1.

In various embodiments of the method described herein, the conjugate pad is prepared by loading at least about 10 μg/mL, at least about 15 μg/mL, at least about 20 μg/mL, at least about 25 μg/mL, at least about 30 μg/mL, or at least about 35 μg/mL of the first anti-coronavirus antibody onto the conjugate pad. In some embodiments of the method described herein, the conjugate pad is prepared by further loading at least about 10 μg/mL, at least about 15 μg/mL, at least about 20 μg/mL, at least about 25 μg/mL, at least about 30 μg/mL, or at least about 35 μg/mL of the first anti-influenzavirus antibody onto the conjugate pad. In one embodiment, the conjugate pad is prepared by loading at least about 15 μg/mL of the first anti-coronavirus antibody onto the conjugate pad. In another embodiment, the conjugate pad is prepared by loading at least about 25 μg/mL of the first anti-coronavirus antibody onto the conjugate pad. In one embodiment, the conjugate pad is prepared by loading at least about 15 μg/mL of the first anti-coronavirus antibody and at least about 15 μg/mL of the first anti-influenzavirus antibody onto the conjugate pad. In another embodiment, the conjugate pad is prepared by loading at least about 25 μg/mL of the first anti-coronavirus antibody and at least about 25 μg/mL of the first anti-influenzavirus antibody onto the conjugate pad.

In certain embodiments described herein, the conjugate pad comprises not more than about 1 μL, about 2 μL, about 5 μL, about 10 μL, or about 20 μL, of OD₂₀ first mobilizable conjugate. In one embodiment, the conjugate pad comprises not more than about 10 μL, of OD₂₀ first mobilizable conjugate. In another embodiment, the conjugate pad comprises not more than about 5 μL, of OD₂₀ first mobilizable conjugate. In many embodiments described herein, the conjugate pad comprises not more than about 1 μL, about 2 μL, about 5 μL, about 10 μL, or about 20 μL, of OD₂₀ second mobilizable conjugate. In one embodiment, the conjugate pad comprises not more than about 10 μL, of OD₂₀ second mobilizable conjugate. In another embodiment, the conjugate pad comprises not more than about 5 μL, of OD₂₀ second mobilizable conjugate. In many embodiments described herein, the conjugate pad comprises not more than about 1 μL, about 2 μL, about 5 μL, about 10 μL, or about 20 μL of OD₂₀ second mobilizable conjugate.

In some embodiments of the method, the second anti-coronavirus antibody is immobilized on the membrane at a concentration of between about 10 μg/mL to 30 μg/mL. In one embodiment of the method, the second anti-coronavirus antibody is immobilized on the membrane at a concentration of about 15 μg/mL. In one embodiment of the method, the first anti-coronavirus antibody is immobilized on the first solid support at a concentration of about 15 μg/mL and the second anti-coronavirus antibody is immobilized on the membrane at a concentration of about 15 μg/mL. In some embodiments of the method, the second anti-coronavirus antibody is immobilized on the membrane at a concentration of between about 10 μg/mL to 30 μg/mL. In one embodiment of the method, the second anti-influenzavirus antibody is immobilized on the membrane at a concentration of about 15 μg/mL. In one embodiment of the method, the first anti-influenzavirus antibody is immobilized on the second solid support at a concentration of about 15 μg/mL and the second anti-influenzavirus antibody is immobilized on the membrane at a concentration of about 15 μg/mL.

In some embodiments of the method, the membrane comprises one or more test zones and one or more control zones, wherein the membrane is selected from the group consisting of nitrocellulose, cellulose acetate, cellulose nitrate, polyamide, polycarbonate, glass fiber, membrane, polyethersulfone, regenerated cellulose (RC), polytetra-fluorethylene, (PTFE), polyester (e.g. Polyethylene Terephthalate), polycarbonate (e.g., 4,4-hydroxy-diphenyl-2,2′-propane), Aluminum Oxide, mixed cellulose ester (e.g., mixture of cellulose acetate and cellulose nitrate), nylon (e.g., Polyamide, Hexamethylene-diamine, and Nylon 66), polypropylene, PVDF, High Density Polyethylene (HDPE)+nucleating agent “aluminum dibenzoate” (DBS). In one embodiment, the membrane is a nitrocellulose membrane. In a specific embodiment, the nitrocellulose membrane is a MDI10μ-CNPF membrane. In another embodiment, the nitrocellulose membrane is a MDI70-CNPH membrane. In various embodiments described herein, the test lines are striped at least about 0.04 μL/mm, at least about 0.08 μL/mm, at least about 0.12 μL/mm with an antibody concentration of about 0.1 mg/mL, about 0.5 mg/mL, about 1.0 mg/mL or about 2.0 mg/mL.

In certain embodiments, the compositions and methods described herein can be used as diagnostic devices that use micro- or macro-array technology in planar or liquid arrays, or that use nitrocellulose, beads, ELISA wells, or other solid support(s). In certain embodiments, the compositions and methods described herein can be used to detect one or more of the following non-limiting embodiments, including all coronaviruses (e.g., using “pan”-specific antibodies) including mutations or novel strains added to the family, and/or each individual coronavirus as described herein. In certain other embodiments, the compositions and methods described herein can be used to detect influenza A/B (for differential diagnosis).

The compositions and methods described herein can use other immunoglobulins, such as immunoglobulins from horse, cattle, pig, sheep, goat, rabbit, guinea pig, rat, mouse or another animal. The antibodies may be monoclonal antibodies, such as a murine, a chimeric, a humanized or a human monoclonal anti-SARS-CoV-2 antibody. In certain embodiments, the compositions and methods described herein can also employ a plurality of a monoclonal antibodies which recognize different epitopes of SARS-CoV-2 antigen. When using monoclonal antibodies, in certain embodiments it may be preferable to employ a mixture of a plurality monoclonal antibodies, in order to broaden the specificity of the assay.

In certain embodiments, the ELISA (enzyme-linked immunosorbent assay) used in accordance with the compositions and methods described herein facilitates the qualitative and quantitative detection of SARS-CoV-2 antigens in fecal samples. In some embodiments, the SARS-CoV-2 antigen is, in a first step, released from the sample and bound by an anti-SARS-CoV-2 antibody, which is immobilized on a solid support (e.g., microtitre plate) or some other solid support. In some embodiments, an additional anti human IgA antibody is added to the assay to capture some or all SARS-CoV-2 epitopes that are already bound by immunoglobulins and are no longer accessible for binding to the first anti-SARS-CoV-2 antibody. In some embodiments, the SARS-CoV-2 antigens in the biological sample (e.g., a fecal sample) are concentrated and bound to the solid phase by the combination of the immobilized anti-SARS-CoV-2 and anti-human IgA antibodies. In the second step, bound SARS-CoV-2 antigens is detected by means of second anti-SARS-CoV-2 conjugated to a detectable agent.

### III. CRISPR-Based Diagnostics

Certain embodiments disclosed herein utilize RNA or DNA targeting effectors to provide a robust CRISPR-based diagnostic with high sensitivity. Embodiments disclosed herein can detect both DNA and RNA with comparable levels of sensitivity and can differentiate targets from non-targets based on single base pair differences. Moreover, the embodiments disclosed herein can be prepared in freeze-dried format for convenient distribution and point-of-care (POC) applications. Such embodiments are useful in multiple scenarios in human health including, for example, viral detection, bacterial strain typing, sensitive genotyping, and detection of disease-associated cell free DNA. In certain embodiments, the present invention is used for rapid detection of viruses using guide RNAs specific to a virus, e.g., a coronavirus or an influenza virus, including, but not limited to 229E, NL63, OC43, HKU1, MERS-CoV, SARS-CoV, SARS-CoV-2, influenzavirus A, influenzavirus B, influenzavirus C, and influenzavirus D.

In one aspect, the embodiments disclosed herein are directed to a nucleic acid detection system comprising a CRISPR system, one or more guide RNAs designed to bind to corresponding target molecules, a reporter construct, and optional amplification reagents to amplify target nucleic acid molecules in a sample. The reporter construct is a molecule that comprises an oligonucleotide component (DNA or RNA) that can be cleaved by an activated CRISPR effector protein. The composition of the oligonucleotide component may be generic i.e. not the same as a target molecule. The reporter construct is configured so that it prevents or masks generation of a detectable positive signal when in the uncleaved configuration, but allows or facilitates generation of a positive detectable signal when cleaved.

Accordingly, in some embodiments, if a target molecule is present in a sample, the corresponding guide molecule will guide the CRSIPR Cas/guide complex to the target molecule by hybridizing with the target molecule, thereby triggering the CRISPR effector protein's nuclease activity. This activated CRISPR effector protein will cleave both the target molecule and then non-specifically cleave the linker portion of the RNA construct.

In another aspect, the embodiments disclosed herein are directed to a lateral flow substrate for detection of one or more viruses (e.g., coronaviruses and/or influenzaviruses). Substrates suitable for use in lateral flow assays may include, but are not necessarily limited to membranes or pads made of cellulose and/or glass fiber, polyesters, nitrocellulose, or absorbent pads (J Saudi Chem Soc 19(6):689-705; 2015). In certain embodiments, one or more CRISPR systems and corresponding reporter constructs are added to the lateral flow substrate at a defined reagent portion of the lateral flow substrate, e.g., on one end of the lateral flow substrate. In some embodiments, reporting constructs used within the context of the present disclosure comprise a first molecule and a second molecule linked by an RNA or DNA linker. The lateral flow substrate further comprises a sample portion. The sample portion may be equivalent to, continuous with, or adjacent to the reagent portion. In some embodiments, the lateral flow strip further comprises a first capture line, typically a horizontal line running across the device, but other configurations are possible. In some embodiments, the first capture region is proximate to and on the same end of the lateral flow substrate as the sample loading portion. In some embodiments, a first capture region comprises a first binding agent.

In some embodiments, the samples to be screened are loaded at the sample loading portion of the lateral flow substrate. In certain embodiments, the samples must be liquid samples or samples dissolved in an appropriate solvent, usually aqueous. In certain embodiments, the liquid sample begins to flow from the sample portion of the substrate towards the first and second (or further) capture regions. If target molecule(s) are present in the sample, the CRISPR effector protein is activated. As activated CRISPR effector protein comes into contact with a bound reporter construct, the reporter construct is cleaved, releasing the detectable reporter construct.

In certain aspects, the invention relates to a method for detecting nucleic acids or polypeptides with a flow strip as defined herein, e.g. (lateral) flow tests or (lateral) flow immunochromatographic assays. In certain example embodiments, a lateral flow device comprises a lateral flow substrate comprising a first end for application of a sample. The first region is loaded with a detectable ligand. The first region also comprises a detection construct. In one example embodiment, a RNA detection construct and a CRISPR effector system (a CRISPR effector protein and one or more guide sequences configured to bind to one or more target sequences). In the presence of target, the CRISPR effector complex forms and the CRISPR effector protein is activated resulting in cleavage of the RNA detection construct (e.g. a guide RNA detection construct). In general, a CRISPR-Cas or CRISPR system as used in herein and in documents, such as WO 2014/093622 (PCT/US2013/074667), refers collectively to transcripts and other elements involved in the expression of or directing the activity of CRISPR-associated (“Cas”) genes.

In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise RNA polynucleotides. The term “target RNA” refers to an RNA polynucleotide being or comprising the target sequence. In other words, the target RNA may be an RNA polynucleotide or a part of an RNA polynucleotide to which a part of the gRNA, i.e. the guide sequence, is designed to have complementarity and to which the effector function mediated by the complex comprising CRISPR effector protein and a gRNA is to be directed. In some embodiments, a target sequence is located in the nucleus or cytoplasm of a cell.

### EXAMPLES

The following examples are put forth so as to provide those of ordinary skill in the art with a description of how the compositions and methods described herein may be used, made, and evaluated, and are intended to be purely exemplary of the present disclosure and are not intended to limit the scope of what the inventors regard as their invention.

### Example 1: Specimen Collection and Preparation

The stool specimen will be collected from patients and placed in an airtight transport container which will be stored at 2-8° C. until they are tested. The stool specimen will be tested as soon as possible, but may be held, e.g., up to 72 hours at 2-8 C prior to testing.

### A. Specimen Preparation

1. Samples will be diluted in an appropriate buffer(s) (e.g., pH 7.2, 10 mM phosphate-buffered solution with 0.02% Thimerosal), referred to herein as a sample diluent. Using a pipetting device, 500 μL of sample diluent will be added to a clean test tube.

2. Stool specimen will be mixed as thoroughly as possible prior to pipetting.

a. Liquid or semi-solid stools—Using the supplied transfer pipette, 100 μL (second mark from the tip of the pipette) of stool will be added into sample diluent. Using same pipette, stool suspension will be mixed gently and thoroughly several times, then are vortexed 15 seconds.

b. Formed/Solid stools—Using a wooden applicator stick, a small portion (5-6 mm diameter) of thoroughly mixed stool is transferred into sample diluent. Emulsified stool is then vortexed 15 seconds.

3. Stool specimens may be centrifuged after dilution at approximately 2750×G for five minutes or until solid matter separates from liquid. The supernatant is recovered and used directly in the ELISA test.

### Example 2: Detection of COVID 19 Antigen in Stool

### A. Coating of the Microtitre Plate

The wells of a microtitre plate are coated with either monoclonal or polyclonal antibodies against COVID 19 antigen and, in some embodiments, human immunoglobulin-A. Approximately 100 μg of polyclonal rabbit-anti-SARS-CoV-2 or murine monoclonal-anti-COVID 19 antibody will be dissolved in 200 μl 160 mM NaCO₃, pH 9.6, and the plate incubated overnight at 4° C. The antibody solution in the wells will be removed and each well will be washed with 200 μl washing buffer (PBS, pH 7.4 with 0.1% Triton X-100). After the last washing procedure, the wells of the microtitre plate are knocked out onto absorbent paper.

### B. Binding Assay

The tests will all be carried out in duplicate. 100 μl standard and sample will be pipetted in duplicate into the antibody coated wells of a microtitre plate and incubated at room temperature for 1 hour while being shaken. The solutions will be removed and the wells of the plate washed five times in each case with 250 μl washing buffer. After the last washing procedure, the microtitre plate will be knocked out dry on absorbent paper.

### C. Detection of Binding

100 μl biotin-conjugated polyclonal rabbit-anti-COVID 19 antibody (1:10000) or horseradish peroxidase (HRP)-conjugated monoclonal-anti-COVID 19 antibody diluted 1:1000 in washing buffer, will be added to the wells, and incubated at room temperature for 1 hour while being vibrated. The solution will be removed from the wells and each well is washed five times with 200 μl washing buffer.

### D. Quantitative Determination

For the color reaction, in the case of horseradish peroxidase-conjugated monoclonal-anti-COVID 19 antibody, 100 μl Tetramethylbenzidin (TMB)-substrate solution (ready-for-use, from NOVUM Diagnostika GmbH, Dietzenbach, Germany) will be dosed into the wells and after about 20 minutes the color development will be stopped by the addition of 50 μl 0.4 M H₂SO₄. In the case of biotin-coupled rabbit-anti-COVID 19 antibody, 100 μl horseradish peroxidase-conjugated streptavidin (DAKO) is coated on, 1:10000 diluted in washing buffer, and incubated for 1 hour at room temperature while being vibrated, washed five times with washing buffer, and only then is the chromogenic substance added. The color development is, in each case, determined by measurement of extinction (optical density) at 450 nm.

### Example 3: Detection of Coronavirus and/or Influenzavirus Antigen(s) in a Sample

### A. Coating of the Microtitre Plate

The wells of a microtitre plate are coated with either monoclonal or polyclonal antibodies against coronavirus and/or influenzavirus antigens and, in some embodiments, human immunoglobulin-A. Approximately 100 μg of polyclonal rabbit-coronavirus and/or anti-influenzavirus or murine monoclonal-anti coronavirus and/or anti-influenzavirus antibody will be dissolved in 200 μl 60 mM NaCO₃, pH 9.6, and the plate incubated overnight at 4° C. The antibody solution in the wells will be removed and each well will be washed with 200 μl washing buffer (PBS, pH 7.4 with 0.1% Triton X-100). After the last washing procedure, the wells of the microtitre plate are knocked out onto absorbent paper.

### B. Binding Assay

The tests will all be carried out in duplicate. 100 μl standard and sample will be pipetted in duplicate into the antibody coated wells of a microtitre plate and incubated at room temperature for 1 hour while being shaken. The solutions will be removed and the wells of the plate washed five times in each case with 250 μl washing buffer. After the last washing procedure, the microtitre plate will be knocked out dry on absorbent paper.

### C. Detection of Binding

100 μl biotin-conjugated polyclonal rabbit-anti-coronavirus and/or anti-influenzavirus antibody (1:10000) or horseradish peroxidase (HRP)-conjugated monoclonal-anti-coronavirus and/or anti-influenzavirus antibody diluted 1:1000 in washing buffer, will be added to the wells, and incubated at room temperature for 1 hour while being vibrated. The solution will be removed from the wells and each well is washed five times with 200 μl washing buffer.

### D. Quantitative Determination

For the color reaction, in the case of horseradish peroxidase-conjugated monoclonal-anti-coronavirus and/or anti-influenzavirus antibody, 100 μl Tetramethylbenzidin (TMB)-substrate solution (ready-for-use, from NOVUM Diagnostika GmbH, Dietzenbach, Germany) will be dosed into the wells and after about 20 minutes the color development will be stopped by the addition of 50 μl 0.4 M H₂SO₄. In the case of biotin-coupled rabbit-anti-coronavirus and/or anti-influenzavirus antibody, 100 μl horseradish peroxidase-conjugated streptavidin (DAKO) is coated on, 1:10000 diluted in washing buffer, and incubated for 1 hour at room temperature while being vibrated, washed five times with washing buffer, and only then is the chromogenic substance added. The color development is, in each case, determined by measurement of extinction (optical density) at 450 nm.

### Example 4: Rapid Detection of SARS-CoV-2 Antigen in Oral Fluid/Saliva Sample

The sandwich-format lateral flow immunoassay is performed with one anti-SARS-CoV-2 IgG antibody conjugated to a reporter particle and a second anti-SARS-CoV-2 antibody immobilized on nitrocellulose membrane to serve as a capture reagent. In this assay, the functionalized reporter particle binds to a specific epitope of nucleocapsid protein in the tested sample and, upon crossing the test line on the lateral flow strip, binds to the capture antibody. The presence of nucleocapsid protein is indicated in this assay by a visible signal of positive detection with the appearance of the test line shown in FIG. 1. Absence of test line signal indicates the absence of nucleocapsid protein in the given sample.

### A. The Sandwich-Format Lateral Flow Immunoassay

The assay is a wet-format test that is composed of a sample pad, conjugate pad, nitrocellulose membrane, and a wick pad (FIG. 1). The nitrocellulose comprises a test line and a control line (FIG. 1). A goat anti-mouse IgG antibody is immobilized at the control line region and an anti-SARS-CoV-2 nucleocapsid protein IgG (MAb clone B3551M) is immobilized at the test line region. A separate anti-SARS-CoV-2 nucleocapsid protein IgG (Arista Biologicals) is conjugated to the surface of gold nanoparticles. Conjugate is placed on the conjugate pad prior to running the assay. Sample (e.g., an oral sample, a mucus sample, a saliva sample and/or a sputum sample) is applied to the sample pad, where it runs through the conjugate pad and travels, with the conjugate, through the nitrocellulose membrane via capillary action (FIG. 2). The assay conditions are outlined in Table 1 below and the test had a sensitivity close to 0.0001 μg/mL in normal pooled saliva (NPS).

TABLE 1
Sandwich-format lateral flow immunoassay conditions

| | Vendor/ | |
|---|---|---|
| Components | (Code) | Conditions |
| Sample Pad | Ahlstrom | 10× PBS [Sigma] pretreatment. |
| | (8950) | 15 mm width |
| Conjugate | Ahlstrom | 10 mm width |
| Pad | (8950) | |
| Nitrocellulose | MDI (10 μ- | Test Line: 0.08 μL/mm dispense rate of |
| | CNPF) | 1.0 mg/mL antibody. |
| Test Line | Meridian | 1.0 mg/mL dispensed at 0.08 μL/mm. |
| Antibody | Bioscience | |
| | (Clone | |
| | B3551M) | |
| Conjugated | ARISTA | 15 μg/mL antibody loading |
| Antibody | Biologicals | 30-minute antibody incubation |
| | | 30-minute blocking incubation |
| Conjugation | N/A | 5 mM Potassium Phosphate with 5 |
| Reaction | | mg/mL PEG20, pH 7.1-7.4 |
| Buffer | | |
| Conjugation | N/A | 4 mM Borate, 1% BSA, 0.05% sodium |
| Block Buffer | | azide |
| Wick Pad | Ahlstrom | 22 mm width |
| | (222) | |

### B. Selection of Antibody Pairs

Seven antibodies were tested for use in the rapid test for detection SARS-CoV-2 nucleocapsid protein. Two mouse monoclonal antibodies were acquired from Meridian Bioscience (clones B3449M and B3551M), whereas four mouse monoclonal antibodies and one rabbit polyclonal antibody were acquired from RayBiotech (clones 130-10785, 1G1-F2, 5F7-A3, 1F9-B5-A5-F10-F11-E6-D3-F11 and 1F2-G1-G1-A12-A3. All the seven antibodies were purified into 10 mM PBS.

Antibodies were striped onto nitrocellulose membranes as test line capture reagents. A goat anti-mouse IgG was striped on the nitrocellulose membranes to serve as a control line. The antibodies were also covalently conjugated to the surface of gold nanoparticles to serve as detection reagents. Testing of the various conjugates with the various antibody test lines allowed for evaluation and selection of antibody pairs.

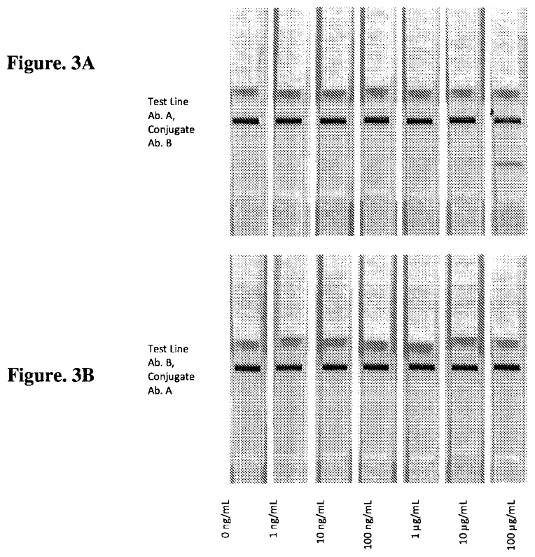

Conjugates prepared with Meridian antibodies were evaluated on test strips containing various antibody test lines. Antibody pairs that resulted in notable levels of non-specific binding (NSB) were excluded from evaluation with contrived samples (Table 2). Conjugates prepared with Raybiotech antibodies were removed from further testing due to significant levels of NSB. The two conjugates prepared with Meridian antibodies exhibited minimal levels of NSB with all potential test line antibodies and were tested with one another as antibody pairs in the assay to select the preferred configuration (FIGS. 3A-B). Nucleocapsid protein was diluted in 10 mM PBS between 1 ng/mL to 100 μg/mL and evaluated with two sandwich assay formats. FIG. 3A shows test strips of the sandwich assay prepared with Conjugate Antibody B (Clone B3451M; Meridian BioScience Catalog No. 9548) and Test Line Antibody A (Clone B3449M; Meridian BioScience Catalog No. 9547). FIG. 3B shows test strips of the sandwich assay prepared with Conjugate Antibody A (Clone B3449M) and Test Line Antibody B (Clone B3451M).

TABLE 2
Selection of antibody pairs

| | Vendor/ | | |
|---|---|---|---|
| | (Code(s)/Clone/ | | |
| Item Description | Part) | Condition(s) | Results |
| Rabbit polyclonal | RayBiotech | Test line antibody, | High NSB observed |
| anti-SARS-CoV-2 | (Code 130- | conjugate antibody | as conjugate. |
| Nucleocapsid Protein | 10785) | | |
| MAb to Nucleocapsid | RayBiotech | Test line antibody, | High NSB observed |
| Protein | (Clone: 1G1-F2) | conjugate antibody | as conjugate. |
| MAb to Nucleocapsid | RayBiotech | Test line antibody, | High NSB observed |
| Protein | (Clone: 5F7-A3) | conjugate antibody | as conjugate. |
| MAb to Nucleocapsid | RayBiotech | Test line antibody, | High NSB observed |
| Protein | (Clone: 1F9-B5- | conjugate antibody | as conjugate. |
| | A5-F10-F11-E6- | | |
| | D3-F11) | | |
| MAb to Nucleocapsid | RayBiotech | Test line antibody, | High NSB observed |
| Protein | (Clone: 1F2-G1- | conjugate antibody | as conjugate. |
| | G1-A12-A3) | | |
| MAb to Nucleocapsid | Meridian | Test line antibody, | Selected for |
| Protein | Bioscience | conjugate antibody | test line |
| | (Clone: | | capture reagent. |
| | B3449M) | | |
| MAb to Nucleocapsid | Meridian | Test line antibody, | Selected as a |
| Protein | Bioscience | conjugate antibody | conjugate antibody. |
| | (Clone: | | |
| | B3551M) | | |
| MAb to Nucleocapsid | ARISTA | Test line antibody, | Selected as a |
| Protein | Biologicals | conjugate antibody | conjugate antibody. |

Contrived samples were prepared by spiking recombinant nucleocapsid protein from Raybiotech into 10 mM PBS at concentrations between 1 to 100 μg/mL. FIGS. 3A-B show the test strip results with affinity reagents with contrived samples. The results in FIG. 3A demonstrated that Antibody B (Clone B3451M) as the detection/conjugate antibody with Antibody A (Clone B3449M) was an antibody pair that exhibited strong signal at 100 μg/mL with minimal NSB. Surprisingly, the reverse sandwich format in FIG. 3B failed to elicit signal at any concentrations evaluated.

### C. Evaluation of Conjugation

Various conjugation conditions with various components were investigated to improve assay performance (Table 3).

TABLE 3
Testing of assay components

| | | | | |
|---|---|---|---|---|
| Item | | | | |
| Description | Supplier | Part/Code | Notes | Results |

Nitrocellulose Membranes

| | | | | |
|---|---|---|---|---|
| MDI70-CNPH | MDI | MDI70-CNPH | Test Lines | Lower signal |
| | | | striped at 0.04, | intensity with |
| | | | 0.08, 0.12 | positive |
| | | | μL/mm for | samples, lower |
| | | | most antibody | NSB. |
| MDI10μ-CNPF | MDI | MDI10μ-CNPF | variants | Strongest signal |
| | | | at ~1.0 mg/mL. | intensity with |
| | | | Control Line | positive |
| | | | striped at 0.08 | samples, higher |
| | | | μL/mm. | NSB. 0.08 |
| | | | | μL/mm |

Sample Pads

| Untreated Pad | Ahlstrom | 8950 | 15 mm, 20 mm | | Fails to reduce |
|---|---|---|---|---|---|
| | | | width evaluated | | NSB. |
| Untreated Pad | Ahlstrom | 8951 | 15 mm width | | Fails to reduce |
| | | | evaluated | | NSB. |
| Pretreated Pad, | Ahlstrom | 8950 | 1) | 10× PBS | 10× PBS |
| 15 mm width | | | | [Sigma] | [Sigma] on |
| | | | 2) | 10× PBS | 8950. |
| | | | | [Thermo | |
| | | | | Fisher] | |
| Pretreated Pad, | Ahlstrom | 8951 | 1) | 10× PBS | 10× PBS |
| 15 mm width | | | | [Sigma] | [Sigma] was |
| | | | 2) | 10× PBS | found to be |
| | | | | [Thermo | preferred. |
| | | | | Fisher] | |

Conjugate Pads

| | | | |
|---|---|---|---|
| Untreated | Ahlstrom | 8950 | 10 mm width |
| Untreated | Ahlstrom | 8951 | 10 mm width |

Wick/Absorbant Pads

| | | | |
|---|---|---|---|
| Grade 222 | Ahlstrom | 222 | 18 mm, 22 mm |
| | | | widths |
| | | | evaluated. |
| Grade 270 | Ahlstrom | 270 | 18 mm |
| | | | evaluated. |

Different amounts of Antibody loading, e.g., 15, 25, and 30 pig/mL (i.e., 15, 25, and 30 μg of antibody per 1 mL of solid support), was evaluated with incubation times of 30, 60, and 120 minutes. Testing of the various conjugates, shown in Table 4, exhibited more favorable results with conjugates prepared at lower antibody loading. The antibody incubation time did not appear to impact assay performance.

TABLE 4
Evaluation of conjugation conditions

| | | | Condition | | |
|---|---|---|---|---|---|
| | Condition | | Evaluated | | |
| Task | Evaluated (1) | | (2- optional) | | Results |
| Reaction | a) | 5 mM Potassium | pH 7.1-7.4 | | 5 mM Potassium |
| Buffer | | Phosphate, 5 | | | Phosphate, 5 mg/mL |
| | | mg/mL PEG 20 | | | PEG20 Reaction buffer |
| | b) | 5 mM Sodium | | | at pH 7.1-7.4 |
| | | Phosphate, 5 | | | identified as a |
| | | mg/mL PEG 20 | | | preferred reaction |
| | | | | | buffer |
| Antibody | a) | 15 μg/mL | >5 | | No observed effect on |
| Loading | b) | 25 μg/mL | Antibodies | | assay performance. |
| | c) | 30 μg/mL | evaluated | | 15 μg of antibody |
| | | | | | per 1 mL solid support |
| | | | | | identified as a preferred |
| | | | | | antibody loading |
| | | | | | concentration |
| Antibody | a) | 15 minutes | N/A | | No observed significant |
| incubation | b) | 30 minutes | | | effect on assay |
| | c) | 60 minutes | | | performance. 30 minutes |
| | d) | 120 minutes | | | identified as a preferred |
| | | | | | antibody incubation time |
| Block | a) | 15 minutes | a) | Blocking | No observed significant |
| Buffer | b) | 30 minutes | | step | effect on assay |
| Incubation | c) | 60 minutes | | included. | performance. 30 minutes |
| | | | b) | No | identified as a preferred |
| | | | | blocking | blocking incubation time |
| | | | | steps. | |

| | | | | |
|---|---|---|---|---|
| EDC | a) | 8 μL per 1 mL | 10 mg/mL | No observed significant |
| Loading | | OD 20 | EDC | effect on assay |
| | b) | 16 μL per 1 mL | solution. | performance. 8 μL per 1 |
| | | OD 20 | | mL OD 20 identified as a |
| | c) | 32 μL per 1 mL | | preferred EDC loading |
| | | OD 20 | | (matrixed with NHS |
| | | | | loading) |
| NHS | a) | 16 μL per 1 mL | 10 mg/mL | No observed significant |
| Loading | | OD 20 | NHS | effect on assay |
| | b) | 32 μL per 1 mL | solution. | performance. 16 μL per 1 |
| | | OD 20 | | mL OD 20 identified as a |
| | c) | 64 μL per 1 mL | | preferred NHS loading |
| | | OD 20 | | (matrixed with EDC |
| | | | | loading) |

Potassium phosphate-based and sodium phosphate-based reaction buffers were evaluated on the impact of conjugate stability. A 5 mM potassium phosphate, 5 mg/mL PEG20 reaction buffer was selected for its favorable impact on conjugate stability throughout the conjugation procedure.

A blocking step was introduced next to evaluate the impact of 4 mM borate, 1% BSA solution on NSB. Addition of the blocking step appeared to improve conjugate stability by reducing the rate at which the conjugate particles settled. This blocking step did not appear to have a significant remedial effect on the NSB, but due to the improved conjugate stability it was included in the conjugation protocol. Additional conditions evaluated were lot-to-lot impact of gold nanoparticles and the volume of EDC and NHS solutions (10 mg/mL) used in the early steps of the conjugation. There did not appear to be lot-to-lot inconsistency on assay performance, nor did the volume variations of EDC and/or NHS appear to impact the final conjugate performance.

A separate antibody supplied by Arista was evaluated as test line and conjugate reagents. The conjugation conditions were 30-minute incubation, 15 μg/mL antibody loading, with a 30-minute blocking step. It was evaluated against Ab A (Clone B3449M) and Ab B (Clone B3451M) in a 3×3 matrix on MDI 10 and MDI 70 membranes with control samples prepared for a separate antigen test. The results demonstrated that performance was better with MDI 10 membranes. Antibody C selected as the conjugate and antibody A (Clone B3449M) was selected as the test line. Antibody B (Clone B3451M) as the detection/conjugate antibody with Antibody A (Clone B3449M)

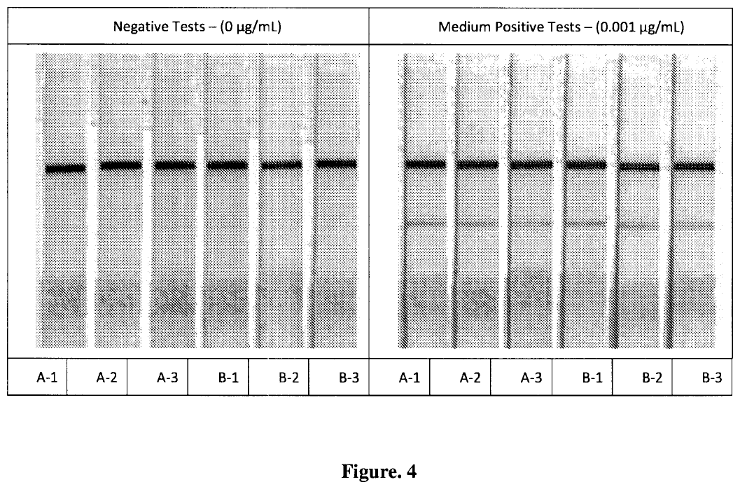

Antibody C conjugation was performed through antibody incubation and antibody loading trials. Conjugations were performed at antibody loadings of 15 and 25 μg/mL (i.e., 15 and 25 μg of antibody per 1 mL of solid support) with incubation periods of 15, 30, and 60 minutes. Testing under these conditions showed no significant difference in assay performance with the various conjugation conditions (FIG. 4).

Additional testing was performed on the conjugate's impact on assay performance. In current liquid format testing, approximately 10 μL of OD 20 conjugate was applied to the test strip and chased with 35-45 μL of sample. When the conjugate volume was increased to 20 μL, a faint increase in test line signal intensity was observe as well as a proportional increase in NSB. These results suggested the increase in conjugate was unnecessary for improvement of the assay performance.

### D. Evaluation of Sample Pads

Saliva/oral fluid samples contain a high concentration of interfering substances that can contribute to non-specific binding. Early development prototype sample pads exhibited significant levels of NSB when run with normal pooled saliva with untreated sample pads. High concentrations of phosphate buffered saline (PBS) can help neutralize the causes of non-specific interactions. Ahlstrom grade 8950 and grade 8951 glass fiber sample pads were pretreated to evaluate performance with normal pooled saliva. 10× phosphate buffered saline (PBS) solutions were prepared from packets acquired through Sigma Aldrich and Thermo Fisher and applied to the various sample pad type variants. Tests prepared with Ahlstrom 8950 sample pads exhibited less NSB than those prepared with 8951 sample pads. Thermo Fisher PBS was removed from efforts after exhibiting elevated levels of NSB, prompting final testing with Sigma PBS pretreatments. The pretreated sample pads appeared to improve the test performance, allowing signal modulation between negative samples and samples spiked to 0.0001 μg/mL nucleocapsid protein (FIG. 5). The results demonstrate that the sample pads described herein demonstrate a competitive sensitivity.

### Other Embodiments

All publications, patents, and patent applications mentioned in this specification are incorporated herein by reference to the same extent as if each independent publication or patent application was specifically and individually indicated to be incorporated by reference.

While the present disclosure has been described in connection with specific embodiments thereof, it will be understood that it is capable of further modifications and this disclosure is intended to cover any variations, uses, or adaptations of the disclosure following, in general, the principles of the disclosure and including such departures from the disclosure that come within known or customary practice within the art to which the disclosure pertains and may be applied to the essential features hereinbefore set forth, and follows in the scope of the claims.

Other embodiments are within the claims.

1-50. (canceled)

51. A rapid detection system for the detection of a coronavirus, or a variant thereof, the system comprising:
a) a sample pad comprising a porous material;
b) a conjugate pad comprising a solid support, wherein a first anti-coronavirus antibody is attached to the solid support to form a mobilizable conjugate; wherein the first anti-coronavirus antibody is capable of binding to a coronavirus antigen to form a mobilizable conjugate-antigen complex; and
c) a membrane comprising a test zone and a control zone, wherein the test zone comprises a second anti-coronavirus antibody immobilized to the membrane, wherein the second anti-coronavirus antibody is capable of binding to the mobilizable conjugate-antigen complex to form a first immobilizable complex, wherein the control zone comprises an immunoglobulin immobilized to the membrane, and wherein the immunoglobulin is capable of binding to the mobilizable conjugate to form a second immobilizable complex.

52. The system of claim 51, wherein the first anti-coronavirus antibody is capable of binding to at least one coronavirus selected from the group consisting of 229E, NL63, OC43, HKU1, MERS-CoV, SARS-CoV and SARS-CoV-2.

53. The system of claim 51, wherein the second anti-coronavirus antibody is capable of binding to at least one coronavirus selected from the group consisting of 229E, NL63, OC43, HKU1, MERS-CoV, SARS-CoV and SARS-CoV-2.

54. The system of claim 51, wherein the coronavirus antigen is selected from the group consisting of a spike protein (S), a receptor-binding (RBD) protein, a S1 protein, a S2 protein, a whole protein (S1+S2), and a nucleocapsid protein (NP).

55-56. (canceled)

57. The system of claim 51, wherein the sample is selected from the group consisting of oral fluid, saliva, blood, blood plasma, blood serum, nasopharyngeal fluid, amniotic fluid, breast milk, vaginal secretions, semen, seminal fluid, urine, amniotic fluid, cerebrospinal fluid, bronchoalveolar lavage fluid, tears, mucous, tissue, tissue homogenate, cellular extract and spinal fluid.

58-72. (canceled)

73. The system of claim 51, wherein the conjugate pad comprises not more than about 10 μL of OD₂₀ mobilizable conjugate.

74. (canceled)

75. A method for rapid detection of a coronavirus, or a variant thereof, in a biological sample, the method comprising
a) dispersing a biological sample suspected of having a coronavirus antigen in a sample buffer;
b) contacting the biological sample dispersed in the sample buffer with a sample pad comprising a porous material through which the biological sample can flow by capillary action;
c) contacting the sample of step b) with a conjugate pad, wherein the conjugate pad comprises a solid support, wherein a first anti-coronavirus antibody is attached to the solid support to form a mobilizable conjugate, wherein the first anti-coronavirus antibody of the mobilizable conjugate is capable of binding to the coronavirus antigen to form a mobilizable conjugate-antigen complex, and wherein the contacting is performed under conditions that permit the capillarity of the mobilizable conjugate; and
d) contacting the mobilizable conjugate-antigen complex with a membrane comprising a test zone, wherein the test zone comprises a second anti-coronavirus antibody immobilized to the membrane, and wherein the second anti-coronavirus antibody is capable of binding to the mobilizable antibody-antigen complex to form a first immobilizable complex; and wherein the first immobilizable complex produces a detectable signal at the test zone indicating the presence of the coronavirus antigen in the biological sample.

76-78. (canceled)

79. The method of claim 75, wherein the coronavirus antigen is selected from the group consisting of a spike protein (S), a receptor-binding (RBD) protein, a S1 protein, a S2 protein, a whole protein (S1+S2), and a nucleocapsid protein (NP).

80-85. (canceled)

86. The method of claim 75, wherein the biological sample is selected from the group consisting of oral fluid, blood, blood plasma, blood serum, nasopharyngeal fluid, amniotic fluid, breast milk, vaginal secretions, semen, seminal fluid, urine, amniotic fluid, cerebrospinal fluid, bronchoalveolar lavage fluid, tears, saliva, mucous, tissue, tissue homogenate, cellular extract, fecal specimen, and spinal fluid.

87-91. (canceled)

92. The method of claim 75, wherein the conjugate pad comprises not more than about 10 μL of OD₂₀ mobilizable conjugate.

93-96. (canceled)

97. A rapid detection system for the detection of a coronavirus and an influenzavirus, comprising:
a) a sample pad comprising a porous material;
b) a conjugate pad comprising a first solid support, wherein a first anti-coronavirus antibody is attached to the first solid support to form a first mobilizable conjugate; wherein the first anti-coronavirus antibody of the first mobilizable conjugate is capable of binding to a coronavirus antigen to form a first mobilizable conjugate-antigen complex, and wherein the conjugate pad further comprises a second solid support, wherein a first anti-influenzavirus antibody is attached to the second solid support to form a second mobilizable conjugate; wherein the first anti-influenzavirus antibody of the second mobilizable conjugate is capable of binding to an influenzavirus antigen to form a second mobilizable conjugate-antigen complex; and
c) a membrane comprising a first test zone, a second test zone, and at least one control zone, wherein the first test zone comprises a second anti-coronavirus antibody immobilized to the membrane, wherein the second anti-coronavirus antibody is capable of binding to the first mobilizable conjugate-antigen complex to form a first immobilizable complex, wherein the second test zone comprises a second anti-influenzavirus antibody immobilized to the membrane, wherein the second anti-influenzavirus antibody is capable of binding to the second mobilizable conjugate-antigen complex to form a second immobilizable complex, wherein the at least one control zone comprises an immunoglobulin immobilized to the membrane, wherein the immunoglobulin is capable of binding to the first mobilizable conjugate to form a third immobilizable complex, and wherein the immunoglobulin is further capable of binding to the second mobilizable conjugate to form a fourth immobilizable complex.

98-100. (canceled)

101. The system of claim 97, wherein the coronavirus antigen is selected from the group consisting of a spike protein (S), a receptor-binding (RBD) protein, a S1 protein, a S2 protein, a whole protein (S1+S2), and a nucleocapsid protein (NP).

102-105. (canceled)

106. The system of claim 97, wherein the sample is selected from the group consisting of oral fluid, saliva, blood, blood plasma, blood serum, nasopharyngeal fluid, amniotic fluid, breast milk, vaginal secretions, semen, seminal fluid, urine, amniotic fluid, cerebrospinal fluid, bronchoalveolar lavage fluid, tears, mucous, tissue, tissue homogenate, cellular extract and spinal fluid.

107-121. (canceled)

122. The system of claim 97, wherein the conjugate pad comprises not more than about 10 μL of OD₂₀ of the first mobilizable conjugate.

123-124. (canceled)

125. The system of claim 97, wherein the conjugate pad comprises not more than about 10 μL of OD₂₀ of the second mobilizable conjugate.

126. (canceled)

127. A method for rapid detection of a coronavirus, or variant thereof, and an influenzavirus, in a biological sample, the method comprising
a) dispersing a biological sample suspected of having either a coronavirus antigen or an influenzavirus antigen, in a sample buffer;
b) contacting the biological sample dispersed in the sample buffer with a sample pad comprising a porous material through which the biological sample can flow by capillary action;
c) contacting the sample of step b) with a conjugate pad, wherein the conjugate pad comprises a first solid support, wherein a first anti-coronavirus antibody is attached to the first solid support to form a first mobilizable conjugate; wherein the first anti-coronavirus antibody of the first mobilizable conjugate is capable of binding to the coronavirus antigen to form a first mobilizable conjugate-antigen complex, wherein the conjugate pad further comprises a second solid support, wherein a first anti-influenzavirus antibody is attached to the second solid support to form a second mobilizable conjugate; wherein the first anti-influenzavirus antibody of the second mobilizable conjugate is capable of binding to the influenzavirus antigen to form a second mobilizable antibody-antigen complex, and wherein the contacting is performed under conditions that permit the capillarity of the first and second mobilizable conjugates;
d) contacting the first mobilizable conjugate-antigen complex with a membrane comprising a test zone, wherein the test zone comprises a second anti-coronavirus antibody immobilized to the membrane, and wherein the second anti-coronavirus antibody is capable of binding to the first mobilizable conjugate-antigen complex to form a first immobilizable complex;
e) contacting the second mobilizable conjugate-antigen complex with the membrane comprising the test zone, wherein the test zone comprises a second anti-influenzavirus antibody immobilized to the membrane, and wherein the second anti-influenzavirus antibody is capable of binding to the second mobilizable conjugate-antigen complex to form a second immobilizable complex; and
wherein the first immobilizable complex produces a first detectable signal indicating the presence of the coronavirus antigen in the biological sample, and wherein the second immobilizable complex produces a second detectable signal indicating the presence of the influenzavirus antigen in the biological sample.

128-133. (canceled)

134. The method of claim 127, wherein the first anti-coronavirus antibody is capable of binding to at least one coronavirus selected from the group consisting of 229E, NL63, OC43, HKU1, MERS-CoV, SARS-CoV and SARS-CoV-2.

135. The method of claim 127, wherein the second anti-coronavirus antibody is capable of binding to at least one coronavirus selected from the group consisting of 229E, NL63, OC43, HKU1, MERS-CoV, SARS-CoV and SARS-CoV-2.

136. The method of claim 127, wherein the coronavirus antigen is selected from the group consisting of a spike protein (S), a receptor-binding (RBD) protein, a S1 protein, a S2 protein, a whole protein (S1+S2), and a nucleocapsid protein (NP).

137-143. (canceled)

144. The method of claim 127, wherein the biological sample is selected from the group consisting of oral fluid, blood, blood plasma, blood serum, nasopharyngeal fluid, amniotic fluid, breast milk, vaginal secretions, semen, seminal fluid, urine, amniotic fluid, cerebrospinal fluid, bronchoalveolar lavage fluid, tears, saliva, mucous, tissue, tissue homogenate, cellular extract, fecal specimen, and spinal fluid.

145-180. (canceled)
